(19) United States
(12) Patent Application Publication
(10) Pub. No.: US 2014/0074176 A1
(43) Pub. Date: Mar. 13, 2014

(54) METHOD AND APPARATUS FOR SELECTIVELY CONTROLLING NEURAL ACTIVITIES AND APPLICATIONS OF SAME

(71) Applicants: Case Western Reserve University, Cleveland, OH (US); Vanderbilt University, Nashville, TN (US)

(72) Inventors: E. Duco Jansen, Nashville, TN (US); Austin Robert Duke, Nashville, TN (US); Michael W. Jenkins, Cleveland, OH (US); Hillel J. Chiel, University Heights, OH (US)

(73) Assignees: Case Western Reserve University, Cleveland, OH (US); Vanderbilt University, Nashville, TN (US)

(21) Appl. No.: 14/024,544

(22) Filed: Sep. 11, 2013

Related U.S. Application Data
(60) Provisional application No. 61/699,735, filed on Sep. 11, 2012.

(51) Int. Cl. A61N 5/06; A61N 1/32

(52) U.S. Cl. CPC A61N 5/0622; A61N 1/32
USPC 607/3; 607/88

(57) ABSTRACT

In one aspect of the present invention, a method of transient and selective suppression of neural activities of a target of interest, such as one or more nerves, includes selectively applying at least one light to the target of interest at selected locations with predetermined radiant exposures to create a localized and selective inhibitory response therein. The localized and selective inhibitory response comprises a local temperature change.

### CROSS-REFERENCE TO RELATED PATENT APPLICATIONS

This application claims priority to and the benefit of, pursuant to 35 U.S.C. §119(e), U.S. provisional patent application Ser. No. 61/699,735, filed Sep. 11, 2012, entitled “OPTICAL INHIBITION OF EXCITABLE TISSUES,” by Austin Robert Duke et al., the disclosure of which is incorporated herein in its entirety by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [14] represents the 14th reference cited in the reference list, namely, A. R. Duke, H. Lu, M. W. Jenkins, H. J. Chiel, E. D. Jansen, Spatial and temporal variability in response to hybrid electro-optical stimulation. J Neural Eng 9, 036003 (Apr. 16, 2012).

### STATEMENT AS TO RIGHTS UNDER FEDERALLY-SPONSORED RESEARCH

This invention was made with government support under grant number CiPHER—HR0011-10-1-0074 awarded by the Department of Defense, and under grant number R01NS052407-01/05 awarded by the National Institutes of Health. The government has certain rights in the invention.

### FIELD OF THE INVENTION

The present invention relates generally to neural stimulations, and more particularly to method and apparatus for selectively controlling neural activities of a target of interest with light, and method for identifying spatial and temporal factors that are controllable for enhancing reproducibility of a hybrid electro-optical stimulation, and applications of the same.

### BACKGROUND OF THE INVENTION

Excitation and inhibition are critical for the normal function of neural circuitry. Thus, to analyze the dynamics of neural circuitry, or to create effective brain-computer interfaces, it is essential to be able to excite or inhibit neurons reversibly and with high specificity. Intracellular microelectrodes make it possible to monitor sub-threshold activity and precisely regulate currents or voltages across the membrane of individual neurons. However, this technology is not practical for large-scale recordings from hundreds of neurons simultaneously, especially in intact, behaving subjects, whose movements will dislodge them, damaging both the electrodes and the neurons. Extracellular electrode arrays provide an effective way to stimulate large numbers of neurons simultaneously, and high frequency electrical stimulation has been developed as a means of inhibiting neurons [1]; but because of current spread, it is often difficult to use these techniques for fine control of individual neurons. At the same time, the burgeoning interest in deep brain stimulation, pain management, functional electrical stimulation, and brain-computer interfaces, have all created a demand for higher levels of specificity and control. In the last decade, optogenetics has become a promising new technology for exciting and inhibiting small groups of neurons with high spatial and temporal precision, but the need for genetic manipulation may create barriers to its clinical use in humans [2, 3].

Several years ago, Wells et al. described the use of infrared laser light to transiently excite neural tissue [4]. Subsequent studies have shown that infrared stimulation works through a spatially precise and thermally-mediated process without the need for genetic modifications [5]. Recent studies have suggested that part of the action of infrared stimulation may be through changes in membrane capacitance [6]. In the last few years, infrared simulation has been used to activate a wide range of excitable tissues including peripheral nerves [4, 7, 8], somatosensory cortex [9], the auditory systems [10], and cardiac tissue [11, 12]. Combining both electrical and infrared stimulation modalities (hybrid electro-optical stimulation) has been shown to be an effective means of both enhancing the specificity of electrical stimulation and reducing the amount of thermal energy that must be deposited in tissue [13, 14].

A recent study by us demonstrated that it was possible to use infrared light to reversibly inhibit excitation of peripheral motor axons, but the mechanism of action was unclear [14]. Other studies had noted that pulsed infrared light could cause inhibitory effects in mammalian cortex, but the process was difficult to control reliably and attributed to activation of inhibitory neurons [9]. Global temperature changes leading to inhibition of action potential generation and propagation, a phenomenon known as “heat block”, have been investigated in both unmyelinated and myelinated preparations [15, 16]. Recent modeling studies indicate the potential for block of action potential generation and propagation with local increases in nerve temperature [17]. The underlying mechanism of global and/or local thermal neural inhibition involves the temperature-dependence of the Hodgkin-Huxley voltage-gated channels. At increased temperatures, the rate of inactivation of sodium channels and activation of potassium channels overwhelms the rate of activation of sodium channels [16-18]. Thus, the recovery phase of the action potential overtakes the rising phase, leading to either a faster and weaker response, or complete but reversible block of the action potential generation or propagation [15, 18].

Hybrid neural stimulation was developed as a new stimulation modality combining traditional electrical techniques with novel infrared nerve stimulation methods [49]. The combination of the two techniques utilizes their respective advantages while avoiding their primary limitations. Specifically, hybrid stimulation combines the safety, established characteristics and demonstrated clinical utility of electrical stimulation with the spatial selectivity of infrared neural stimulation (INS). While hybrid stimulation does not provide the contact- and artifact-free aspects of INS, the high spatial selectivity of INS remains and enhances clinical neural interfaces. Additionally, sub-threshold electrical currents should also reduce the problem of electrode corrosion over time. The essence of hybrid stimulation is to combine a sub-threshold electrical stimulus over a broad area, and then bring a spatially selective location to threshold by adding a sub-threshold pulse of infrared light. In doing so, both the electrical current and optical radiant exposures are reduced, effectively achieving spatial selectivity with reduced risk of tissue damage. Previously, hybrid stimulation was shown to reduce optical radiant exposures (J cm⁻²) by approximately a factor of 3 when compared to INS alone [49]. By offering reduced threshold radiant exposures, hybrid nerve stimulation is attractive for biomedical applications requiring spatial selectivity where laser power constraints and tissue damage are primary concerns. However, further development of this technology requires that the reliability and repeatability of hybrid stimulation be improved.

The experiments demonstrating feasibility of hybrid stimulation in the rat sciatic nerve showed large variations in the reduction of optical radiant exposures [49]. In these experiments, the electrical threshold was set at a chosen sub-threshold current and the additional optical radiant exposure required to achieve stimulation threshold was determined as a percent of the optical threshold radiant exposure when it was applied alone. The reduction in optical radiant exposures and their variability were both shown to increase as the applied electrical stimulus approached threshold. For an electrical stimulus at 95% of the threshold current, the additional optical energy required for stimulation ranged from 6% to 60% of the optical stimulation threshold.

Hybrid electro-optical neural stimulation is a novel paradigm combining the advantages of optical and electrical stimulation techniques while reducing their respective limitations. However, in order to fulfill its promise, this technique requires reduced variability and improved reproducibility.

Therefore, a heretofore unaddressed need exists in the art to address the aforementioned deficiencies and inadequacies.

### SUMMARY OF THE INVENTION

In one aspect, this invention involves the use of optical techniques for inhibiting activity in excitable tissues or target endpoints controlled by the excitable tissue. In embodiments of the invention, infrared wavelengths are used to inhibit neural activity. However, the invention is not constrained to infrared wavelengths or neural applications. This invention works in endogenous tissues, which is fundamentally different from optogenetic techniques that require genetic modifications to allow optical control. The underlying mechanism of this invention is proposed to be a thermally mediated process, whereby a sufficient temperature increase in the excitable tissue changes the rate at which ion channels are opened and closed. While global temperature changes in neurons leading to block of action potential generation and propagation has been known for decades, the invention demonstrates the use of light to create a local temperature change for selective and reversible inhibition. According to the invention, this technology can be used to improve the selectivity of electrical stimulation and to block propagating action potentials away from their site of generation.

In one aspect, the present invention relates to a method of transient and selective suppression of neural activities of a target of interest. The target of interest contains one or more nerves of a living subject, such a human or animal. In one embodiment, the method includes selectively applying at least one light to the target of interest at selected locations with predetermined radiant exposures to create a localized and selective inhibitory response therein. In one embodiment, the localized and selective inhibitory response comprises a local temperature change.

In one embodiment, the neural activities comprise generation and propagation of action potentials. The action potentials are evoked electrically by an electrical stimulus applied to the target of interest.

In one embodiment, the at least one light comprises pulses of a single light generated from a laser source.

In one embodiment, the pulses of the single light are synchronized with the electrical stimulus, such that the pulses of the single light and the electrical stimulus end at the same time.

In another embodiment, the pulses of the single light are applied prior to the start time of the electrical stimulus at a first predetermined time.

In yet another embodiment, the pulses of the single light are applied after the start time of the electrical stimulus at a second predetermined time.

In one embodiment, the at least one light comprises two or more lights, and each of the two or more lights comprises pulses of light generated from a respective laser source.

In one embodiment, the pulses of the two or more lights are synchronized with the electrical stimulus, such that the pulses of the two or more lights and the electrical stimulus end at the same time.

In another embodiment, the pulses of the two or more lights are applied prior to the start time of the electrical stimulus at a first predetermined time.

In yet another embodiment, the pulses of the two or more lights are applied after the start time of the electrical stimulus at a second predetermined time.

In one embodiment, the step of selectively applying the at least one light to the target of interest comprises simultaneously applying the two or more lights to the target of interest at the selected locations,

In another embodiment, the step of selectively applying the at least one light to the target of interest comprises alternately or sequentially applying the two or more lights to the target of interest at the selected locations.

In one embodiment, each of the at least one light comprises an infrared light.

In another aspect, the invention relates to an apparatus for selectively controlling of neural activities of a target of interest. In one embodiment, the apparatus has a source for generating at least one light; and a probe coupled to the at least one light source for selectively delivering the at least one light to the target of interest at selected locations to create a localized and selective inhibitory response therein.

In one embodiment, the neural activities comprise generation and propagation of action potentials. In one embodiment, the action potentials are evoked electrically by an electrical stimulus applied to the target of interest.

In one embodiment, the light source comprises a laser source, and the at least one light comprises pulses of a single light generated from the laser source.

In one embodiment, the pulses of the single light are synchronized with the electrical stimulus, such that the pulses of the single light and the electrical stimulus end at the same time.

In another embodiment, the pulses of the single light are applied prior to the start time of the electrical stimulus at a first predetermined time.

In a further embodiment, the pulses of the single light are applied after the start time of the electrical stimulus at a second predetermined time.

In one embodiment, the light source comprises two or more light laser sources, and the at least one light comprises two or more lights, each light comprising pulses of light generated from a respective laser source of the two or more light laser sources.

In one embodiment, the pulses of the two or more lights are synchronized with the electrical stimulus, such that the pulses of the two or more lights and the electrical stimulus end at the same time.

In another embodiment, the pulses of the two or more lights are applied prior to the start time of the electrical stimulus at a first predetermined time.

In yet another embodiment, the pulses of the two or more lights are applied after the start time of the electrical stimulus at a second predetermined time.

In one embodiment, the probe is configured to simultaneously deliver the two or more lights to the target of interest at the selected locations,

In another embodiment, the probe is configured to alternately or sequentially deliver the two or more lights to the target of interest at the selected locations.

In one embodiment, each of the at least one light comprises an infrared light.

In one embodiment, the probe comprises at least one optical fiber having one end coupled to the at least light source and a working end positioned proximate to the target of interest for selectively delivering the at least one light to the target of interest at the selected locations.

In yet another aspect, the invention relates to a method for identifying spatial factors that are controllable for enhancing reproducibility of a hybrid electro-optical stimulation to a target of interest. In one embodiment, the method includes simultaneously applying electrical pulses at a sub-threshold and optical pulses of a set magnitudes to the target of interest, wherein the optical pulses of a set magnitudes are delivered by an optical fiber; translating the optical fiber back and forth across the target of interest, and measuring a position of the optical fiber when translating; reconstructing the exact position of the optical fiber at the time of the hybrid stimulation; and correlating the working end of the optical fiber with the presence or absence of the hybrid stimulation as indicated by an evoked potential on a nerve recording, so as to obtain the spatial factors.

In one embodiment, The method of claim 33, wherein the sub-threshold is about 90% less than the threshold of the electrical stimulation.

In one embodiment, the method further includes determining existence of a finite region of excitability (ROE) with size altered by the strength of the optical stimulus and recruitment dictated by the polarity of the electrical stimulus.

In one embodiment, the electrical pulses and the optical pulses are synchronized such that they end concurrently.

In a further aspect, the invention relates to a method for identifying temporal factors that are controllable for enhancing reproducibility of a hybrid electro-optical stimulation to a target of interest. In one embodiment, the method includes simultaneously applying electrical pulses and optical pulses to the target of interest; regularly measuring threshold currents of the electrical stimulus to monitor underlying changes in the electrical stimulation with time, and measuring radiant exposures eliciting the hybrid stimulation along with the threshold currents of the electrical stimulus; reducing the stimulus current to a sub-threshold; applying different radiant exposures along with the sub-threshold current pulses to the target of interest, and recording each hybrid stimulus pulse as either a 1 or 0 as determined by the presence (1) or absence (0) of action potentials; repeating the process for the predetermined duration; and processing the recorded data to obtain the temporal factors.

In one embodiment, the electrical pulses and the optical pulses are synchronized such that they end concurrently.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows infrared inhibition of action potential initiation. (A) A micropipette providing supra-threshold extracellular electrical stimulation is flanked by two optical fibers transverse to the longitudinal axis of BN2. Extracellular nerve recordings are obtained from the three branches distal to trifurcation. (B) Schematic representation of the nerve cross-section at the site of thermal inhibition. Axons are arranged in hypothetical locations consistent with the observed results. (C) Neural recordings from branches of BN2 showing selective inhibition (arrows) of action potential generation. Each laser inhibits the generation of an action potential projecting to a single nerve branch. Upon removal of the infrared pulse, electrically evoked action potentials return, indicating reversibility. (D) Neural recordings from branches of BN2 showing combined inhibition of two nerve branches. By applying infrared pulses from both lasers simultaneously, nerve responses projecting to BN2b and BN2c are inhibited (arrows), while electrically evoked action potentials projecting to BN2a are unaffected. (E) Average iCNAP recorded from each nerve branch in response to electrical only, electrical plus Laser 1 and electrical plus Laser 2; **p<0.01 (N=3 nerves; n=5 trials).

FIG. 2 shows infrared inhibition of propagating action potentials in BN2 of Aplysia. (A) A micropipette electrically stimulated action potentials that propagated to the three branches of BN2. A 200 μm diameter optical fiber coupled to a diode laser source provided infrared pulses distal to the site of electrical stimulation and proximal to the nerve trifurcation. (B1) A train of infrared pulses (λ=1450 nm; τₚ=0.2 msec; indicated schematically by a gray bar) at 200 Hz inhibits the propagation of action potentials projecting to BN2c (inhibited responses are highlighted by a yellow bar). A single spontaneous response is evident on the BN2a recording (arrow). This was occasionally observed during laser application as well as before and/or after. Action potentials on BN2b show slight inhibition on this recording, but were not statistically significant (p>0.05) across all samples. Electrical artifacts have been blanked for clarity. (B2) Evoked and inhibited responses are shown at the beginning of infrared inhibition and immediately following the infrared pulse train (arrows). (C) Average iCNAP for response immediately preceding and following the infrared stimulus train, as well as the first inhibited response; *** p<0.001 (N=3 nerves; n=11 trials.

FIG. 3 shows infrared inhibition of electrically evoked muscle contraction. (A) A suction electrode stimulated the nerve to induce muscle contractions in the I1/I3 muscles as measured by a force transducer. A 200 μm diameter optical fiber placed distal to the electrical stimulus inhibited action potential propagation of some of the motor units. (B1) Electrically evoked force in response to five 2-sec stimuli at 10 Hz. (B2) In the same preparation, a 3-sec infrared pulse train (λ=1450 nm; τₚ=0.2 msec) at 200 Hz delivered in conjunction with the third electrical stimulus inhibited force generation. (C) Average I1/I3 contraction force in response to electrical stimulation with and without the infrared pulse train; ***p<0.001 (without laser, n=5 trials; with laser, n=5 trials).

FIG. 4 shows infrared inhibition of propagating action potentials in the rat sciatic nerve. (A) A monopolar cuff electrode stimulated propagating action potentials along the main nerve trunk. A 400 μm diameter fiber optic coupled to a diode laser source was positioned over the tibial branch of the nerve. A train of infrared pulses (λ=1450 nm; τₚ=0.2 msec; indicated schematically by a gray bar) at 200 Hz reduces the amplitude of EMG recordings for MG and LG. Electrical artifacts have been blanked for clarity. (B2) Evoked and reduced EMG responses are shown at before, during and after infrared inhibition. (C) Average iEMG normalized to the iEMG value for evoked responses before infrared inhibition; *** p<0.001 (N=2 nerves; n=12 trials).

FIG. 5 shows an effect of relative pulse timing on threshold radiant exposures for inhibition. Infrared pulses (τₚ=0.25 msec) delivered up to 10 msec before a supra-threshold electrical stimulus (τₚ=0.25 msec) will consistently inhibit action potential initiation, though threshold radiant exposures for inhibition are higher than for shorter delay intervals Inhibiting radiant exposures increase sharply when the infrared pulse is delivered after the electrical stimulus (* p<0.05 compared to t=−0.25 msec; N=2 nerves; n=4 trials).

FIG. 6 shows a nerve temperature increase during infrared inhibition. (A) Temperature was measured using a thermal imaging camera positioned above the nerve preparation. (B) Using parameters previously found to block action potential propagation, the nerve temperature rises by approximately 8° C. Thermal relaxation (i.e., the time required for the temperature to fall to 1/e of baseline) is approximately 80 msec.

FIG. 7 shows titration of muscle force inhibition. Infrared inhibition is capable of titrating electrically evoked force. By decreasing the radiant exposure, less of the muscle force is inhibited. (A) A suction electrode stimulates the nerve to induce muscle contractions in the I1/I3 muscles as measured by a force transducer. A 200 μm diameter optical fiber placed distal to the electrical stimulus inhibits action potential propagation along motor units. (B1) Electrically evoked force in response to five 2-sec stimuli at 10 Hz. (B2) In the same preparation, a 3-sec infrared pulse train (λ=1450 nm; τₚ=0.2 msec) at 200 Hz delivered in conjunction with the third electrical stimulus inhibits force generation. (C) Average I1/I3 contraction force in response to electrical stimulation with and without the infrared pulse train; *p<0.05 (without laser, n=5 trials; with laser, n=5 trials).

FIG. 8 shows an evoked muscle movement in response to infrared thermal inhibition. Using a video of the muscle movement, pixel shift for points located at ventral, medial and dorsal positions on the I1/I3 muscle were determined in response to electrical stimulation with and without infrared thermal inhibition. The medial portion of the muscle consistently experiences less movement in response to infrared thermal inhibition, whereas the ventral portion shows increased movement. Of the trials shown (n=2), electrical stimulation plus infrared thermal inhibition resulted in increased movement of the dorsal portion of the muscle for one trial and less movement in the other.

FIG. 9 shows infrared pulses can enhance propagated responses in the rat sciatic nerve. EMG recordings from MG increase in peak-to-peak amplitude during the infrared pulse train. Following infrared pulses, the EMG responses begin to return to their pre-infrared exposure magnitudes. EMG recordings from LG are unchanged in response to infrared pulses. Electrical stimulation artifacts have been blanked for clarity.

FIG. 10 shows experimental setups used for the (A) Aplysia californica buccal nerve (50×) and (B) rat sciatic nerve (20×) experiments in this study. RN=radular nerve; CBC=cerebrobuccal connective; BN3=buccal nerve 3; BN2=buccal nerve 2; BN1=buccal nerve 1; EN=esophageal nerve.

FIG. 11 shows evaluation of an output of system. To evaluate electrical, optical and hybrid stimulation, we looked for the presence of single and/or compound extracellular nerve potentials in the Aplysia californica buccal nerve and single and/or compound muscle potentials in the innervated muscles of the rat sciatic nerve. A representative recording from (A) the Aplysia californica buccal nerve and (B) the innervated muscle (biceps femoris) of the rat sciatic nerve.

FIG. 12 shows (A) A finite ROE exists between the cathode and anode where the combination of sub-threshold electrical and optical stimuli will achieve neural activation in an Aplysia nerve. Outside of this ROE, stimulation does not occur. (B) Evoked electrical response to hybrid stimulation recorded from the distal nerve. (C) Absence of evoked response outside of ROE. Hybrid stimulus parameters used: 675 μA (100 μs), 4.58 J/cm² (3 ms). Electrical stimulation threshold was 750 μA. In (B) and (C), the LED and electrical stimulation artifacts are indicated by the shaded region.

FIG. 13 shows a finite ROE exists between the cathode and anode where the combination of sub-threshold electrical and optical stimuli will achieve neural activation. ROEs for the Capella and Ho:YAG within the same Aplysia nerve are shown in (A) and (B), respectively. Typical ROEs observed in the rat sciatic nerve are shown for the Capella (c) and Ho:YAG (D).

FIG. 14 shows an ROE size as a function of radiant exposure in the buccal nerve of Aplysia californica (A)-(C) and the rat sciatic nerve (D)-(F).

FIG. 15 shows changing the polarity of a sub-threshold electrical stimulus (90% of electrical stimulation threshold) in the Aplysia buccal nerve yields two distinct regions of excitability (ROEs) with both the (a) Capella (λ=1.875 μm; τₚ=3 ms; H=4.97 J/cm²) and (b) Ho:YAG (λ=2.120 μm; τₚ=0.25 ms; H=2.67 J/cm²) lasers. The location of the ROE is adjacent to the location of the cathode. The dark-colored circles represent locations of successful hybrid stimulation when the cathode is located on the left side of the nerve. The light-colored circles represent locations of successful hybrid stimulation when the polarity is reversed and the cathode is located on the right side of the nerve.

FIG. 16 shows electrical stimulation threshold and REM) for hybrid stimulation as a function of time in an Aplysia californica buccal nerve. (A) Results from one nerve showing a negative correlation (r²=−0.47, p<0.05) between thresholds for electrical stimulation and the RE₅₀ for hybrid stimulation measured every 2 min. (B) Probability of firing as a function of radiant exposure using data accumulated from all animals. The slope of the CDF fit at 50% probability indicates the amount of variability in hybrid stimulation radiant exposures yielding stimulation over time. Effects of adjusting the electrical priming current every 2 min versus every 20 min are also shown. More frequent adjustments to the priming current increase the slope of the CDF fit, thus reducing variability in threshold radiant exposure for the optical component of hybrid stimulation. Note that the y-intercept for the 20 min adjustment plot is greater than 0, suggesting that there is a small probability of firing even with 0 J/cm² of optical stimulus. This is due to rare occasions where the electrical stimulation threshold fell below the previously set sub-threshold stimulus before the next adjustment was made.

FIG. 17 shows electrical stimulation threshold and REM) for hybrid stimulation as a function of time in the rat sciatic nerve. (A) Results from one nerve showing a negative correlation (r²=−0.66, p<0.05) between threshold for electrical stimulation and the REM) for hybrid stimulation measured every 2 min. (B) Probability of firing as a function of radiant exposure in each animal using all data acquired over 1 hr. The slope of the CDF fit at 50% probability indicates the amount of variability in threshold measurements over time. There is more variability between animals in the rat than in Aplysia (FIG. 16B).

FIG. 18 shows a limited window of radiant exposures for successful hybrid stimulation in Aplysia. A >50% probability of firing with an electrical stimulus at 90% of electrical stimulation threshold requires radiant exposures from 1.34 to 4.79 J/cm². Evoked responses to a range of radiant exposures were acquired every 2 min for 1 h. These data were aggregated to achieve a probability of firing for each radiant exposure. The increasing and decreasing phases of the plot were then each fitted to a CDF.

FIG. 19 shows an optical stimulation of sufficient radiant exposure will inhibit electrically evoked action potentials. In both (A) and (B), a supra-threshold stimulus (110% of threshold) is applied (100 μs, 567 μA). In (A), the optical stimulus (3 ms) is 5.73 J/cm², whereas in (B), the optical stimulus is 6.49 J/cm2. Note how the electrically evoked action potential is present in (A) but not in (B). The electrical stimulation artifact is indicated by the shaded region.

FIG. 20 shows results of hybrid inhibition in which for a constant electrical stimulus there is a window of optical energies for which hybrid stimulation occurs according to embodiments of the invention.

FIG. 21 shows curves of threshold current vs. temperature for hybrid stimulation and infrared inhibition according to embodiments of the invention.

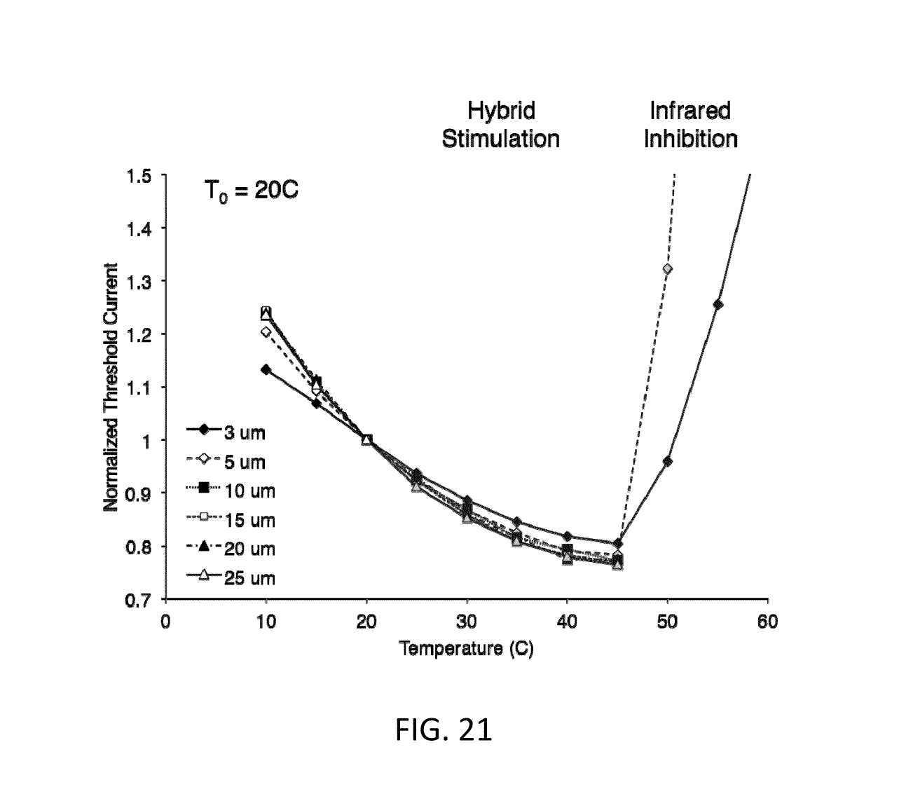

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, “plurality” means two or more. As used herein, the terms “comprising”, “including”, “carrying”, “having”, “containing”, “involving”, and the like are to be understood to be open-ended, i.e., to mean including but not limited to.

As used herein, the term “inhibition” refers to a transient elimination of action potential initiation or generation, while the term “block”” refers to a transient impediment to action potential propagation.

### OVERVIEW OF THE INVENTION

In one aspect, this invention involves the use of optical techniques for inhibiting activity in excitable tissues or target endpoints controlled by the excitable tissue. In embodiments of the invention, infrared wavelengths are used to inhibit neural activity. However, the invention is not constrained to infrared wavelengths or neural applications. One embodiment of this invention works in endogenous tissues, which is fundamentally different from optogenetic techniques that require genetic modifications to allow optical control. The underlying mechanism of this invention is due to a thermally mediated process, whereby a sufficient temperature increase in the excitable tissue changes the rate at which ion channels are opened and closed. While global temperature changes in neurons leading to block of action potential generation and propagation has been known for decades, the invention demonstrates the use of light to create a local temperature change for selective and reversible inhibition. According to the invention, this technology can be used to improve the selectivity of electrical stimulation and to block propagating action potentials away from their site of generation.

The primary novel element of this invention is the use of light to create a localized and selective inhibitory response. The application of this local inhibition to enhance current interfaces or to control unwanted activity is also novel.

The invention addresses two primary problems. (1) Current interfaces with excitable tissues are limited in their ability to selectively recruit sub-populations spatially and, in the case of neurons, following the physiological recruitment order of smallest neurons before largest neurons. Using light, one is able to selectively inhibit the activation of sub-populations of excitable tissues, thereby enhancing the selectivity of the method used for stimulation. (2) There are many clinical and research applications where it is desirable to block unwanted activity. The invention allows selective block of propagating biopotentials to prevent them from reaching their endpoint. For example, this would allow for titrated control of sensory perception or block of spastic neuromuscular activity.

Potential products and applications of this technology include peripheral nerve interfaces (e.g. nerve cuff), brain-computer interfaces, combination with high-frequency electrical nerve conduction block, control of cardiac function, pain management, functional neuromuscular stimulation, cochlear implants, analysis of neural circuitry and dynamics.

In another aspect, the invention relates to method for identifying spatial and temporal factors that play a role in and are controlled to enhance the reproducibility of hybrid electro-optical stimulation.

The hybrid electro-optical neural stimulation that combines the advantages of optical and electrical stimulation techniques while reducing their respective limitations. However, in order to fulfill its promise, this technique requires reduced variability and improved reproducibility. According to the invention, a comparative physiological approach is used to aid the further development of this technique by identifying the spatial and temporal factors characteristic of hybrid stimulation that may contribute to experimental variability and/or a lack of reproducibility. Using transient pulses of infrared light delivered simultaneously with a bipolar electrical stimulus in either the marine mollusk Aplysia californica buccal nerve or the rat sciatic nerve, we determined the existence of a finite region of excitability with size altered by the strength of the optical stimulus and recruitment dictated by the polarity of the electrical stimulus. Hybrid stimulation radiant exposures yielding 50% probability of firing (REM) were shown to be negatively correlated with the underlying changes in electrical stimulation threshold over time. In Aplysia, but not in the rat sciatic nerve, increasing optical radiant exposures (J cm⁻²) beyond the REM ultimately resulted in inhibition of evoked potentials. Accounting for the sources of variability identified in this study increased the reproducibility of stimulation from 35% to 93% in Aplysia and 23% to 76% in the rat with reduced variability.

These and other aspects of the present invention are more specifically described below.

### IMPLEMENTATIONS AND EXAMPLES OF THE INVENTION

Without intent to limit the scope of the invention, exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example One

### Infrared Control of Electrically Activated Neurons

This example demonstrates that, among other things, infrared light can precisely turn off electrically stimulated neurons. Specifically, pulses of infrared light can be utilized to reversibly inhibit action potential generation and propagation with high temporal and spatial specificity, and to reversibly control functional output, i.e., muscle force. These results could provide the basis for novel techniques for studying neural circuitry, and for selectively controlling peripheral neuronal activity, which could have significant implications for the development of more precise brain-computer interfaces and prosthetic devices.

A detailed investigation was carried out using the unmyelinated buccal nerve 2 (BN2) of the marine mollusk Aplysia californica buccal ganglion. This nerve provides a robust and experimentally tractable ex vivo preparation with substantial length and a distal trifurcation that allows for simultaneous recording of multiple branches, and a muscular target that is known and tractable to study. These results were also validated in the myelinated rat sciatic nerve.

### Materials and Methods

### Aplysia Preparation and Electrophysiology

Aplysia californica (n=4) weighing 250-350 g (Marinus Scientific, Long Beach, Calif.) were maintained in an aerated aquarium containing circulating artificial seawater (ASW) (Instant Ocean; Aquarium Systems, Mentor, Ohio) kept at 16-17° C. The animals were fed dried seaweed every 1-3 days.

Aplysia were anesthetized with an injection of 333 mM MgCl₂ (˜50% of body weight) prior to dissection. Once anesthetized, animals were dissected and the buccal ganglia were removed and pinned in a recording dish and immersed in Aplysia saline (460 mM NaCl, 10 mM KCl, 22 mM MgCl₂, 33 mM MgSO₄, 10 mM CaCl₂, 10 mM glucose, 10 mM HEPES, pH 7.6). Aplysia buccal ganglia are symmetric, so each hemiganglion has an associated buccal nerve 2 (BN2). Each BN2 was transected just distal to its attachment to its respective hemiganglion and anchored in place by pinning the protective sheath around the nerve to the Sylgard base (Dow Corning, Midland, Mich.) of the recording dish. Once securely pinned, the three distal branches of BN2 were suctioned into nerve-recording electrodes to monitor the response to stimulation. Nerve-recording electrodes were made by hand-pulling polyethylene tubing (1.27 mm outer diameter, 0.86 mm inner diameter; PE90; Becton Dickinson) over a flame to the desired inner diameter. Recording electrodes were suction-filled with Aplysia saline prior to suctioning of the nerve. Nerve signals were amplified (×1000) and band-pass filtered (300-500 Hz) using an AC-coupled differential amplifier (model 1700; A-M Systems), digitized (Axon Digidata 1320A; Molecular Devices, Sunnyvale, Calif.) and recorded (Axograph X; Axograph Scientific).

Extracellular stimulating electrodes were made from thin-wall borosilicate capillary glass (catalogue No. 6150; A-M Systems, Everett, Wash.) pulled to a diameter of about 40 μm and resistances of about 0.1 MΩ (model P-80/PC; Sutter Instruments, Novato, Calif.). For each experiment, an electrode was capillary-filled with Aplysia saline and positioned on the top surface of the nerve, in contact with the nerve sheath, using a micromanipulator. The return electrode was positioned at a distance in the bath to create monopolar stimulation. Monophasic currents supplied by a stimulus isolator (A360; WPI) were used for all experiments.

### Delivery of Infrared Light to Nerves

Two tunable diode laser systems were used throughout the study in this example. Laser 1 includes a prototype tunable diode laser (Capella; Lockheed-Martin-Aculight, Bothwell, Wash.) with wavelength λ=1450 nm coupled to a 200 μm diameter fiber optic (Ocean Optics, Dunedin, Fla.). Laser 2 includes a similar and commercially available diode laser (λ=1860 nm) coupled to a 100 μm diameter fiber optic. Fiber optics was secured in place using micromanipulators.

### Data Acquisition and Analysis

Amplified and filtered nerve responses were acquired at 5 kHz. AxoGraph X software (AxoGraph X; AxoGraph Scientific, Sydney, Australia) was used to coordinate stimulation and inhibition protocols, and to record acquired data. Post-acquisition data analysis was performed using a combination of AxoGraph X, Matlab (Matlab r2010b; Mathworks, Natick, Mass.) and Microsoft Excel (part of Microsoft Office Professional Plus 2010). Data are expressed as mean plus/minus the standard error of the mean.

### Radiant Exposure Determination

Radiant exposures normalize applied optical energy per unit area. Radiant energy was measured using an energy meter and pyroelectric energy detector (Nova II, Ophir; PE50BB-VR-ROHS, Ophir). The radiant exposure was determined by dividing the radiant energy by the area of the circular fiber tip (i.e., 0.0314 mm²). In order to report radiant exposure at the level of the axons, many assumptions and calculations would be necessary. For simplicity and accuracy, the measured value at the tip of the fiber optic prior to any additional assumptions was used.

For the rat, the laser spot size incident on the nerve surface was measured using the knife-edge technique. Thus, we report a measured spot-size (0.0026 cm²) to provide greater accuracy. These methods of radiant exposure determination are consistent with published literature.

### Infrared Inhibition of Action Potential Generation

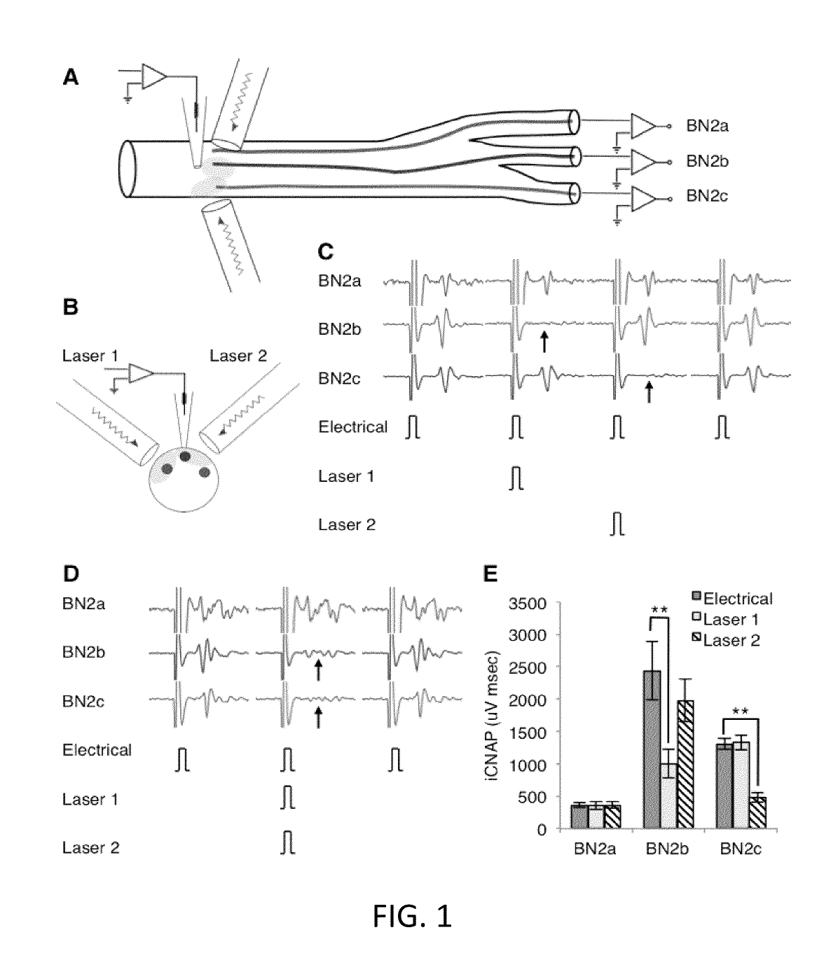

Fiber optics from laser systems 1 and 2 were positioned such that they flanked the stimulating electrode transverse to the nerve's longitudinal axis as shown FIG. 1A. Parameters used for each laser system are shown in Table 1 below. Differences in pulse durations and fiber optic diameters used were due to laser power constraints and nerve working area. The 1450 nm laser used in these experiments produces five times the power of the 1860 nm laser (25 W and 5 W, respectively), and is thus capable of operating at lower pulse durations. The discrepancy in required radiant exposures can be attributed to the difference in absorption for the wavelengths used. The absorption of infrared light in tissue can be approximated by the absorption of infrared light by water [5]. The absorption coefficient of water at 1860 nm (μₐ=12.8 cm⁻¹) is roughly 2.5 times less than at 1450 nm (μₐ=32.7 cm⁻¹) [31]. Thus, greater radiant exposures must be provided at 1860 nm to generate the same overall absorption and associated temperature increase as at 1450 nm. Threshold radiant exposures for inhibition at 1450 nm are similar to the prior observations in Aplysia using a diode laser operating at 1875 nm [14]. Infrared absorption at 1875 nm (μₐ≈26 cm⁻) is much closer to that of 1450 nm, further confirming that the wavelength is not as critical as absorption and thermal conversion of light in tissue. To verify that differences in laser sources, pulse durations and fiber diameters did not play a role in the results, a limited set of experiments was performed where the 1450 nm laser was set to a constant pulse duration and alternately coupled to either of two 200 μm diameter fibers that were positioned on either side of the micropipette. Results from this limited study (data not shown) demonstrated that the results shown in FIG. 1 were not an artifact of the use of two laser systems.

Each trial (n=5) included a series of repeating 500 msec episodes. For each episode, a monophasic electrical stimulus (τₚ=0.25 msec) providing current sufficient to generate consistent action potentials on all three recording electrodes (461.4±36.2 μA) was applied at 100 msec. Pulses of infrared inhibition from each laser source were synchronized with the supra-threshold electrical stimulus such that the pulses ended at the same time. This allowed total charge and total heat deposition to occur simultaneously. Each trial typically followed an ABACABACA pattern in which nerves were stimulated electrically (A), then either Laser 1 or Laser 2 was added (B), then the laser was removed leaving only electrical stimulation (A), followed by the other laser being added (C), and then the process was repeated. Nerve responses for each condition were analyzed using the integrated compound nerve action potential (iCNAP): the ensemble average for each condition within a given trial was rectified and summed over 20 msec following the electrical stimulation artifact.

TABLE 1
Parameters of Lasers 1 and 2 for Inhibition
of Action Potential Generation.

| | Wave- | Absorption | Fiber | Pulse | |
|---|---|---|---|---|---|
| | length - | Coefficient in | Optic | Duration - | Radiant |
| Laser | λ | H₂O (31) - μₐ | Diameter | τₚ | Exposure - H |
| 1 | 1450 nm | 32.7 cm⁻¹ | 200 μm | 0.5 msec | 4.43 ± 0.30 |
| | | | | | J/cm² |
| 2 | 1860 nm | 12.8 cm⁻¹ | 100 μm | 5 msec | 8.34 ± 0.78 |
| | | | | | J/cm² |

### Effect of Relative Pulse Timing on Infrared Inhibition

To characterize how the relative timing of the infrared and electrical pulses affects threshold radiant exposures for inhibition of action potential generation, a single infrared pulse (λ=1450 nm, τₚ=0.5 msec) was delivered at time points before and after an electrical stimulus (τₚ=0.25 msec). The timing scheme was such that t=0 corresponded to the infrared and electrical pulses ending simultaneously. The infrared pulse was delivered over the range of t=−20 msec to t=0.5 msec (n=4 for each time point). For each trial, the electrical stimulus was 110% of the threshold current, where electrical threshold was defined as the minimum current required to generate 5 consecutive evoked responses. Infrared pulses (n=10) at 5 different radiant exposures were applied for each time point. The presence (1) or absence (0) of an evoked response was recorded and aggregated to achieve the probability of a stimulated response for each radiant exposure. At each time point, the probability versus radiant exposure data is fit to the negative of the cumulative distribution function (CDF). Threshold for infrared thermal inhibition at each time point was defined as the radiant exposure generating <50% of an evoked response [14].

### Infrared Inhibition of Action Potential Propagation

The nerve preparation was as described previously, except a single 200 μm fiber optic coupled to the 1450 nm laser source was positioned approximately 1 cm distal to the site of electrical stimulation, but proximal to the nerve trifurcation (FIG. 1B). Each trial (n=11) included one 10 sec episode. Monophasic electrical stimuli (τₚ=0.25 msec; 659.1±18.9 μA) providing consistent responses on all three branches of BN2 were delivered at 4 Hz for the duration of the trial. At 4 sec, pulses of infrared light (τₚ=0.2 msec) were delivered at 200 Hz for 3 seconds. Nerve responses were analyzed using the iCNAP as described above.

### Nerve Temperature

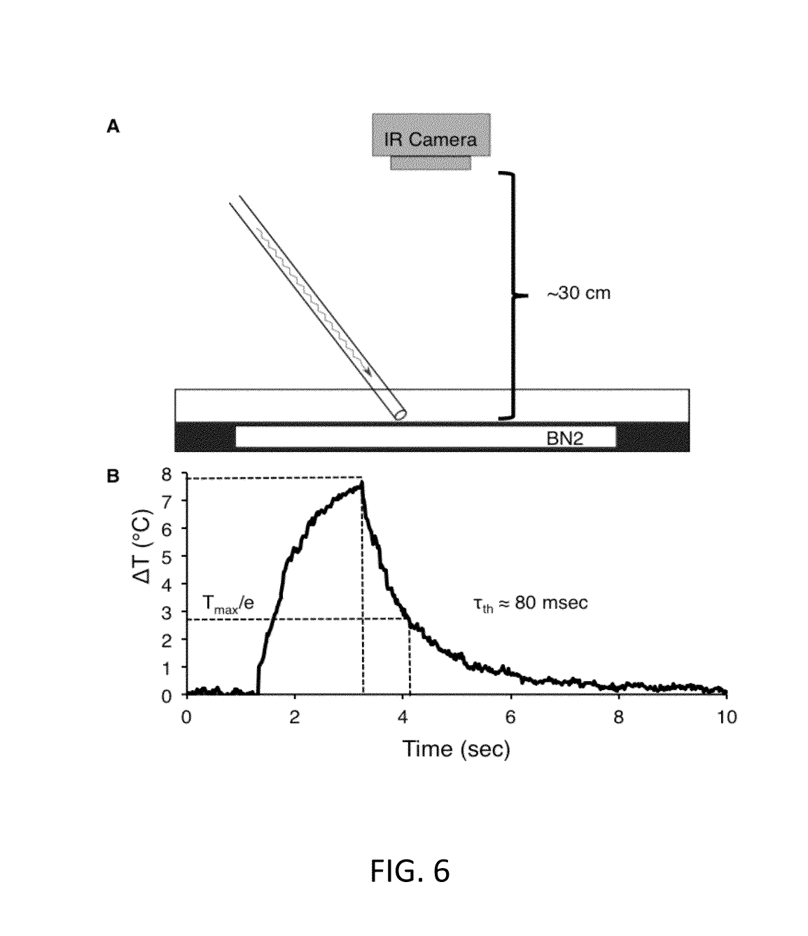

BN2 of an Aplysia (314 g) was dissected and secured to a recording dish. The saline level of the Sylgard-covered dish was lowered so that it was just covering the surface of the nerve (FIG. 6A). A 200 μm fiber optic coupled to the 1450 nm laser was positioned above the nerve such that the tip of the fiber was just out of contact with the nerve. Infrared pulses (0.52 J/cm²) were delivered at 200 Hz for 3 sec. A thermal imaging camera (FLIR Systems Thermovision A20) was positioned approximately 30 cm above the nerve. Images were acquired at 60 Hz for 25 seconds. Rat temperature measurements were made using the same setup while applying infrared pulses (τp=0.2 msec, 0.12 mJ/cm², 200 Hz) to the rat sciatic nerve in vivo.

To find the temperature change required for nerve conduction block in Aplysia, we averaged all trials (N=3 nerves, n=11 trials) and found the minimum duration of laser exposure for which the BN2c iCNAP was significantly reduced. Significance was determined using p<0.004 in Aplysia and p<0.002 in the rat after correcting for multiple comparisons using the Bonferroni method. This duration was then compared to the measured temperature (FIG. 6B) to determine the induced temperature rise. The same procedure was applied to the rat, where minimum infrared exposure duration required to significantly reduce the iEMG for LG was determined and compared to the measured temperature change.

### Muscle Force Measurements

An Aplysia (422 g) was anesthetized with an injection of approximately 50% body weight isotonic MgCl₂. The animal's buccal mass was removed and placed in a Petri dish within a solution of 50% Aplysia saline and 50% isotonic MgCl₂. Both buccal nerves 2 were severed at their attachment points to the buccal ganglia. Incisions were made through the dorsal and ventral surfaces of the buccal mass, and further incisions were made to remove the radula-odontophore and pharyngeal tissue, leaving the I1/I3 muscle split into two separate halves with each half innervated by its buccal nerve 2. The rest of the buccal mass and the ganglia were discarded. The muscle halves were moved to a recording dish with a Sylgard surface in the back half of the dish. Each I1/I3 half was glued (Duro Quick-Gel superglue, Henkel Corp., Avon, Ohio) by its anterior edge to the glass bottom of the dish just in front of the Sylgard. After gluing, the dish was filled with Aplysia saline. Each buccal nerve 2 was gently stretched and pinned on the Sylgard surface, and polyethylene suction electrodes were attached to the ends of the nerves. A 200 μm diameter fiber optic coupled to the 1450 nm laser source was positioned distal to the suction electrode and proximal to the nerve trifurcation. Force transducers (Grass Technologies, West Warwick, R.I.) were attached to the medial portions of the I1/I3 halves using silk sutures.

Electrical stimulation was applied using the nerve suction electrodes. Control trials included 5 repetitions of electrical stimulation (τₚ=1 msec, 500 μA) delivered at 10 Hz for 2 sec. Each repetition was followed by an interval of 12 seconds with no stimulation. Experimental trials included the same protocol. In addition, however, infrared pulses (τₚ=0.2 msec) were applied at 200 Hz for 3 seconds beginning 1 second before the third electrical stimulus. Five sets of control and experimental trials were repeated for a given parameter set with 3 min between each trial to allow the nerve to rest.

### Infrared Inhibition of Action Potential Propagation in a Rat

All experiments were performed following protocols approved by the Institutional Animal Care and Use Committee (IACUC). Male Sprague-Dawley rats (n=2) weighing 250-300 g (Charles River) were anesthetized with continuously inhaled isoflurane (induction: 3% isoflurane, 3.0 LPM oxygen; maintenance: 2-2.5% isoflurane, 1.5 LPM oxygen). A rectal probe and heating pad (catalog No. 40-90-8, FHC, Bowdoin, Me.) were used to maintain the rat at a target body temperature of 35-37° C. throughout the experiment. The animals were placed on a polycarbonate platform and their hindlimbs were shaved. The dorsal surface of the foot was then taped to the edge of the platform. An incision was made from the heel to the vertebral column and the skin was separated from the underlying tissue. The biceps femoris was then cut and divided proximal from the Achilles tendon to expose the sciatic nerve. The sural and peroneal branches of the sciatic nerve were transected so only innervation of the planterflexor muscles remained.

Paired EMG electrodes made from perfluoroalkoxy (PFA)-coated silver wire (0.003″ bare, 0.005″ coated; A-M Systems, Sequim, Wash.) were inserted along the length of the medial gastrocnemius and lateral gastrocnemius muscles. EMG signals were amplified (×100) and band-pass filtered (100-1000 Hz) using an AC-coupled differential amplifier (model 1700; A-M Systems), digitized (20 kHz; Axon Digidata 1440A; Molecular Devices, Sunnyvale, Calif.) and recorded (Axograph X; Axograph Scientific).

A monopolar nerve cuff electrode was placed around the trunk of the sciatic nerve. Each trial (n=12) included one 10 sec episode. Monophasic electrical stimuli (τₚ=0.1 msec; 750 μA) were delivered at 8 Hz for the duration of the trial. At 4 sec, pulses of infrared light (τₚ=0.2 msec; 75.7 mJ/cm²) were delivered at 200 Hz for 3 seconds. Laser spot size for radiant exposure calculations was measured using the knife-edge technique [32]. Nerve responses were analyzed using the iEMG, which was calculated in the same manner as the iCNAP described above.

### Results and Discussions

### Infrared Inhibition of Action Potential Generation

To investigate the selective inhibition of electrically evoked action potentials, an extracellular micropipette was used to provide nonspecific supra-threshold stimulation to the main trunk of BN2. Electrically evoked responses were recorded on the three distal branches of BN2: BN2a, BN2b and BN2c [19, 20], allowing the primary compound nerve action potential to be deconvolved and resolved into some of its spatial components. Two optical fibers were positioned on opposite sides of the micropipette and coupled to independent laser sources (FIG. 1A). Laser 1 includes a tunable diode laser with wavelength λ=1450 nm and pulse duration τₚ=0.25 msec coupled to a 200 μm diameter fiber optic. Laser 2 includes a similar diode laser (λ=1860 nm, τₚ=5 msec) coupled to a 100 μm diameter fiber optic. By synchronizing the electrical stimulus with a pulse of infrared light from a single laser source, one was able to selectively inhibit the initiation of an action potential that ordinarily appeared in one branch of BN2. Alternating between laser sources demonstrated that each blocked the initiation of a different electrically evoked response (FIG. 1C). When both lasers provided a pulse of infrared light simultaneously, responses on two of the branches were inhibited, while an electrically activated response remained largely unchanged on the third branch (FIG. 1D). Removing the infrared pulses unblocked the electrically evoked response on all three branches, indicating that this selective inhibition is completely reversible. In most cases, larger units were primarily inhibited, though smaller units were preferentially blocked in some cases. Increasing the radiant exposure (J/cm²) resulted in inhibition of a larger population or of all units (data not shown). The integrated compound nerve action potential (iCNAP) was used as a metric for the level of electrical activation for each branch (FIG. 1E). Reduction in the iCNAP of BN2b (p<0.01) was observed when Laser 1 provided infrared pulses, whereas reduction in the iCNAP of BN2c (p<0.01) occurred as a result of Laser 2 providing the infrared pulses (N=3 nerves; n=5 trials). No change in the iCNAP of BN2a was observed when either or both lasers were used. The application of a single infrared pulse capable of affecting only one branch of BN2 suggests that interactions between optical energy and the micropipette are not the primary underlying mechanism of this phenomenon. Although electrode effects cannot be completely excluded, if the interaction of light and pipette altered current densities at the pipette-nerve interface, one would expect a change in the responses measured on all three branches. While different wavelengths were used in these experiments (1860 vs. 1450 nm), it was demonstrated that this combination was not essential for observing these results.

The results obtained in the example indicate some amount of selective inhibition in both location and size of axons. At this time, it is not clear why infrared inhibition predominantly blocked action potential propagation along BN2c without significantly affecting BN2a or BN2b. In some trials, action potentials on BN2b experienced an increase in size during infrared inhibition of BN2c, while in other a slight decrease was observed. These affects were not statistically significant (p>0.05). These results may imply neurophysiological differences in the axonal units projecting to the different branches of BN2. Motor neurons are known to project to BN2c [33]. Unpublished data from our lab imply that BN2b and BN2c together contain the axons of the motor neurons, while BN2a contains sensory neuronal projections. Evoked responses at the soma of motor neurons were found to project to BN2b and/or BN2c, but not BN2a. This conclusion is further suggested by the observed lack of I1/I3 muscle contraction when both BN2b and BN2c are severed and BN2a is left intact. Published studies also show that stimulation of BN2a directly leads to activity in interneurons B4/B5 [19] as well as the elicitation of motor programs [20]. In addition to neurophysiological differences in projected neurons, it is also possible that projections to BN2c are more peripherally located relative to the nerve cross-section. This would allow the infrared-induced temperature gradient to reach axons projecting to BN2c before affecting those projecting to BN2a or BN2b. In the case of increased magnitude of action potentials on BN2b, this may be due to lower temperatures at the periphery of the laser spot size inducing hybrid electro-optical stimulation as opposed to inhibition [5, 34].

### Effect of Relative Pulse Timing on Infrared Inhibition

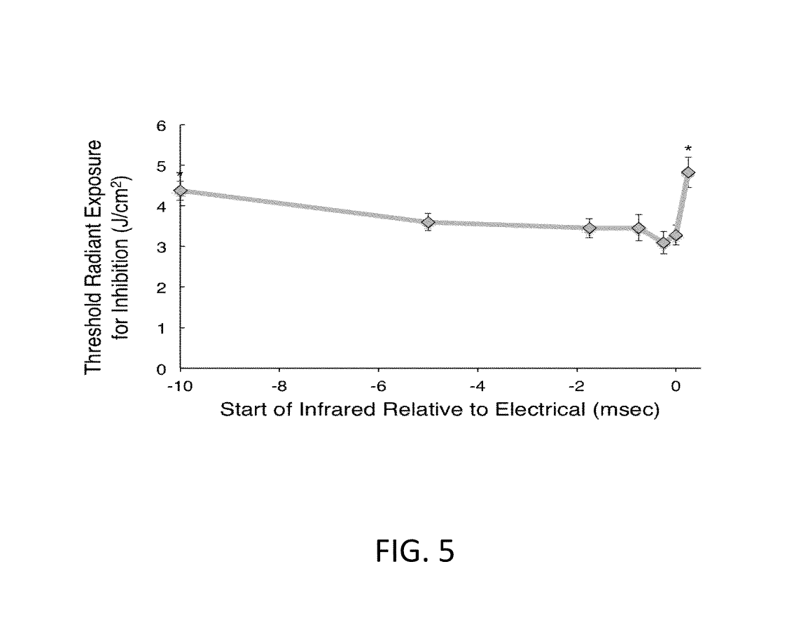

To characterize how the relative timing of the infrared and electrical pulses affects threshold radiant exposures for inhibition of action potential generation, a single infrared pulse (λ=1450 nm, τₚ=0.5 msec) was delivered at time points before and after an electrical stimulus (τₚ=0.25 msec). With the infrared pulse (τₚ=0.25 msec) delivered prior to the electrical pulse (τₚ=0.25 msec), threshold radiant exposures for inhibition slowly increased as the timing between the pulses increased (FIG. 5). Inhibition reliably occurred with the infrared pulse delivered as much as 10 msec prior to the start of the electrical pulse. Threshold radiant exposures for inhibition rapidly increased when the infrared pulse was delivered after the electrical stimulus. Inhibition occurred reliably with the infrared pulse delayed 0.25 msec after the electrical pulse, but was not observed with the infrared pulse delayed by 0.5 msec. Minimum threshold radiant exposures for inhibition occurred when the infrared pulse was delivered 0.25 msec before the electrical pulse.

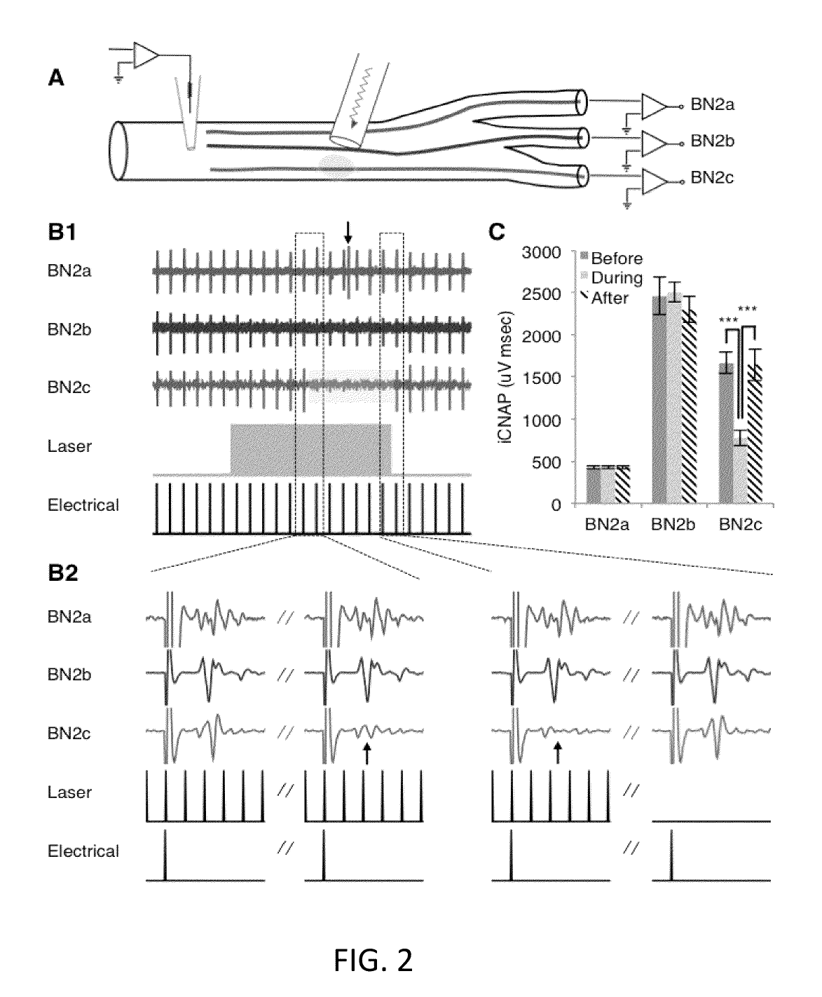

Infrared and electrical pulses were synchronized for the purpose of demonstrating temporally precise inhibition of action potential initiation (FIG. 1). However, FIG. 5 indicates that there is a narrow temporal window for which the infrared pulse can be applied to induce inhibition. For the experimental preparation, if the infrared pulse was applied >10 msec before or >0.25 msec after the electrical stimulus, inhibition would not reliably occur before infrared stimulation threshold was reached. While some studies and applications will utilize precise tracking of infrared and electrical pulses for block of action potential initiation, many applications could benefit from a high frequency train of infrared pulses, as demonstrated in FIG. 2.

### Infrared Inhibition of Action Potential Propagation

The nerve preparation was as described previously, except a single 200 μm fiber optic coupled to the 1450 nm laser source was positioned approximately 1 cm distal to the site of electrical stimulation, but proximal to the nerve trifurcation (FIG. 1B). Each trial (n=11) includes one 10 sec episode. Monophasic electrical stimuli (τₚ=0.25 msec; 659.1±18.9 μA) providing consistent responses on all three branches of BN2 were delivered at 4 Hz for the duration of the trial. At 4 sec, pulses of infrared light (τₚ=0.2 msec) were delivered at 200 Hz for 3 seconds. Nerve responses were analyzed using the iCNAP as described above.

In addition to inhibiting the initiation of electrically evoked action potentials, localized block of propagating responses was also demonstrated. Electrically evoked responses were stimulated at 4 Hz and propagated to BN2a, BN2b and BN2c. A single fiber optic was positioned along the nerve trunk distal to the site of supra-threshold electrical stimulation (at about 1 cm) (FIG. 2A) and a 3-second train of low intensity (0.50±0.02 J/cm²), high frequency (200 Hz) infrared pulses was applied to produce a smooth rise in local tissue temperature without sharp peaks (FIG. 6B). At sufficient optical intensity, the block of a response projecting to BN2c was observed (FIG. 2B1). The magnitude of the iCNAP for BN2c during the propagation block was lower (p<0.001; N=3 nerves; n=11 trials) than the magnitude of the response just before and just after the infrared train (FIG. 2C). Blocked propagation usually began during the second half of the infrared pulse train. At higher radiant exposures, increased spontaneous activity on BN2a and/or BN2b was observed.

The temperature measured in this study for the Aplysia is an overestimate of the actual temperature reaching BN2. In order to visualize temperature with the IR camera the fiber optic was kept above the surface of the saline/nerve rather than immersed in the saline as during experimentation. Thus, an insulating saline-air interface was present during temperature measurements. When modeling laser-tissue interactions, the tissue-air interface is often considered adiabatic with heat reflecting back into the simulated volume [35]. In the actual experimental preparation, the added saline above the site of infrared absorption would help to conduct heat away and yield a lesser temperature rise than was measured with the IR camera.

The tissue temperature in response to infrared inhibition of propagation action potentials was also measured in the rat. A supra-threshold stimulus (0.12 mJ/cm²) resulted in an approximately 10° C. increase in tissue temperature.

### Muscle Force Measurements

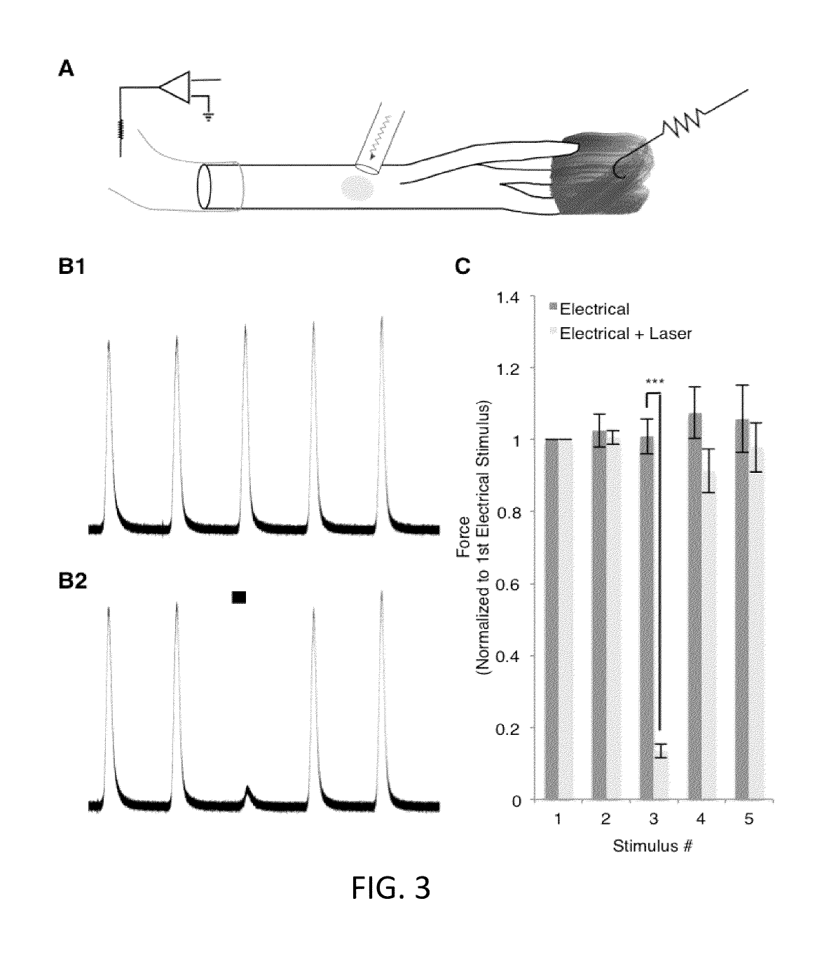

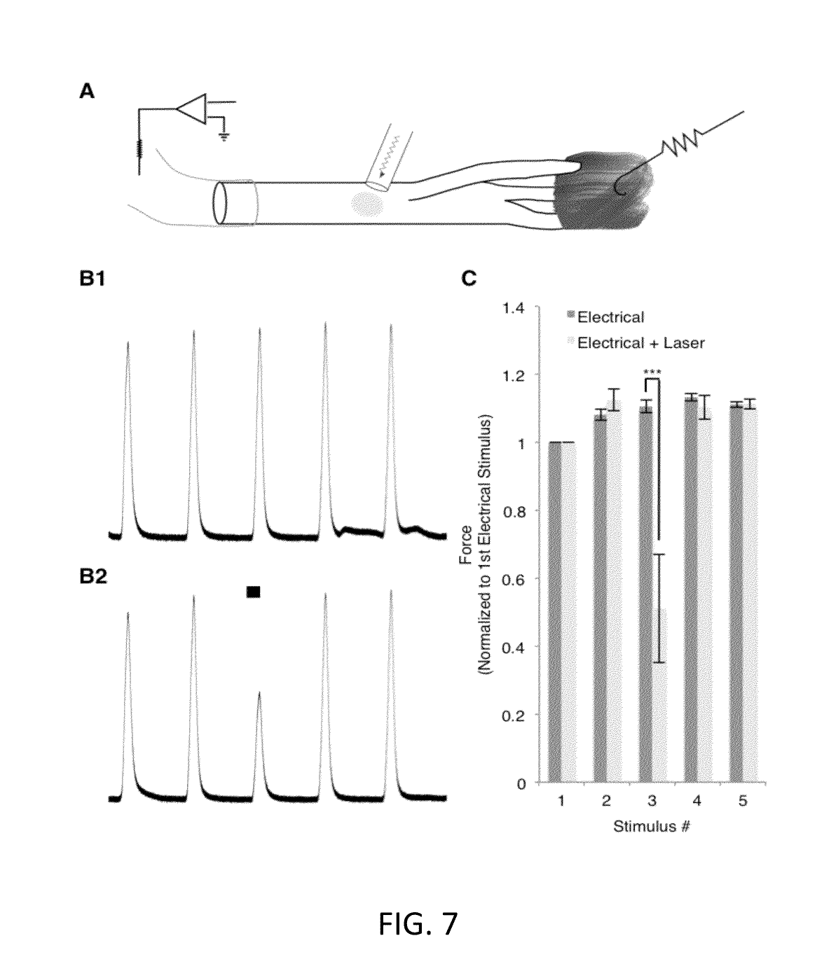

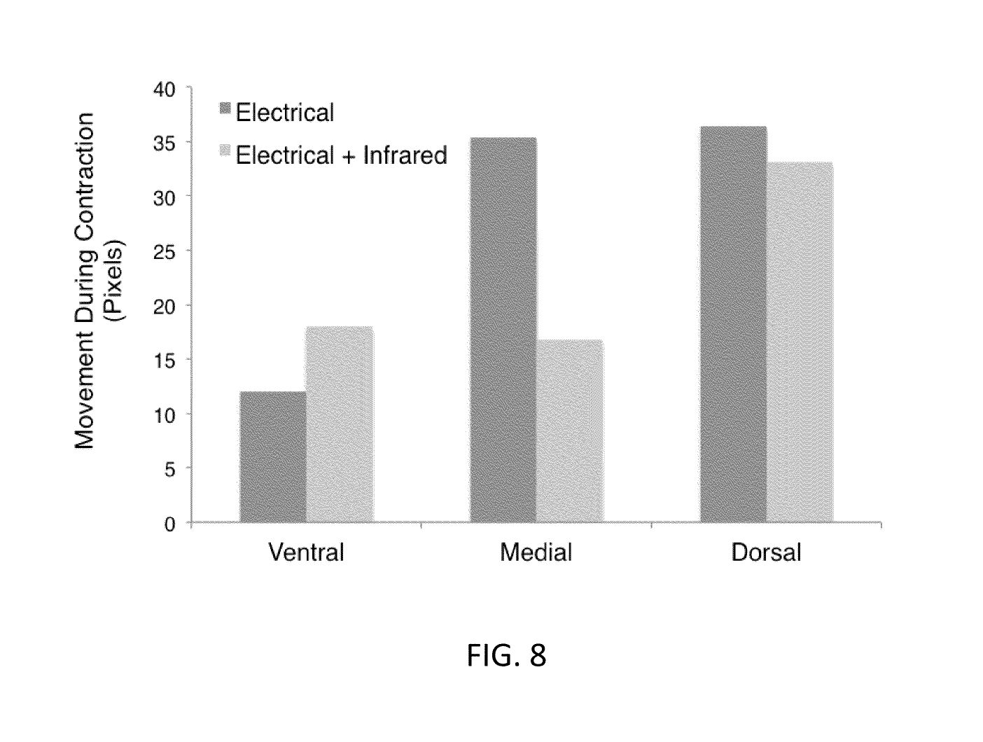

To demonstrate the functional relevance of the inhibition, the effects on muscle force were measured. The distal BN2 muscle innervation was left intact and the contraction force of the I1/I3 muscles was measured with a force transducer (FIG. 3A). As a control, five repetitions of electrical stimulation (2 sec, 10 Hz) were applied with 12 sec between each stimulus (FIG. 3B1). When the infrared pulses (3 sec, 200 Hz) were applied beginning 1 second before the third electrical stimulus, measured forces were reduced (FIG. 3B2). The addition of infrared pulses significantly reduced the force produced (p<0.001; without laser, n=5 trials; with laser, n=5 trials) by nearly 90% when compared to the preceding and following electrically generated forces (FIG. 3C). The reduction in generated force could be titrated by adjusting the radiant exposure of the infrared pulses or changing the location of the fiber optic relative to the center of the nerve (FIG. 7). Preliminary results indicate that inhibition of part of the motor pool affects contraction in a specific muscle region (FIG. 8).

### Selectivity of Inhibition and Enhancement in the Rat Sciatic Nerve

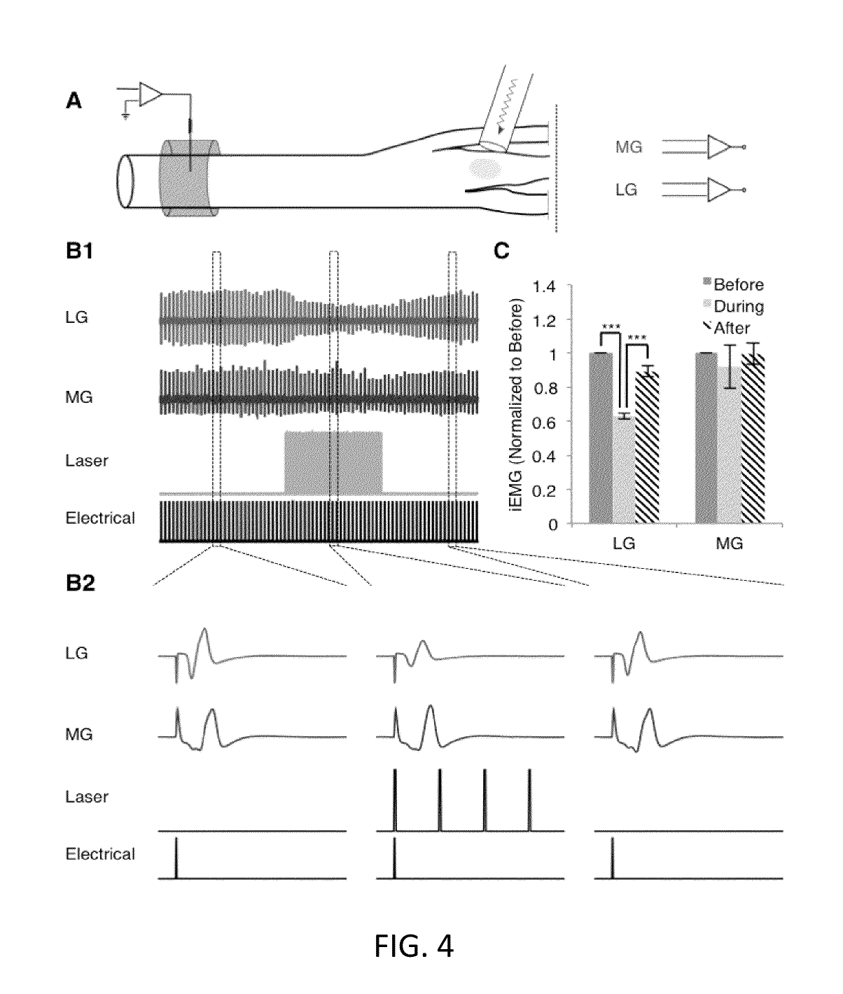

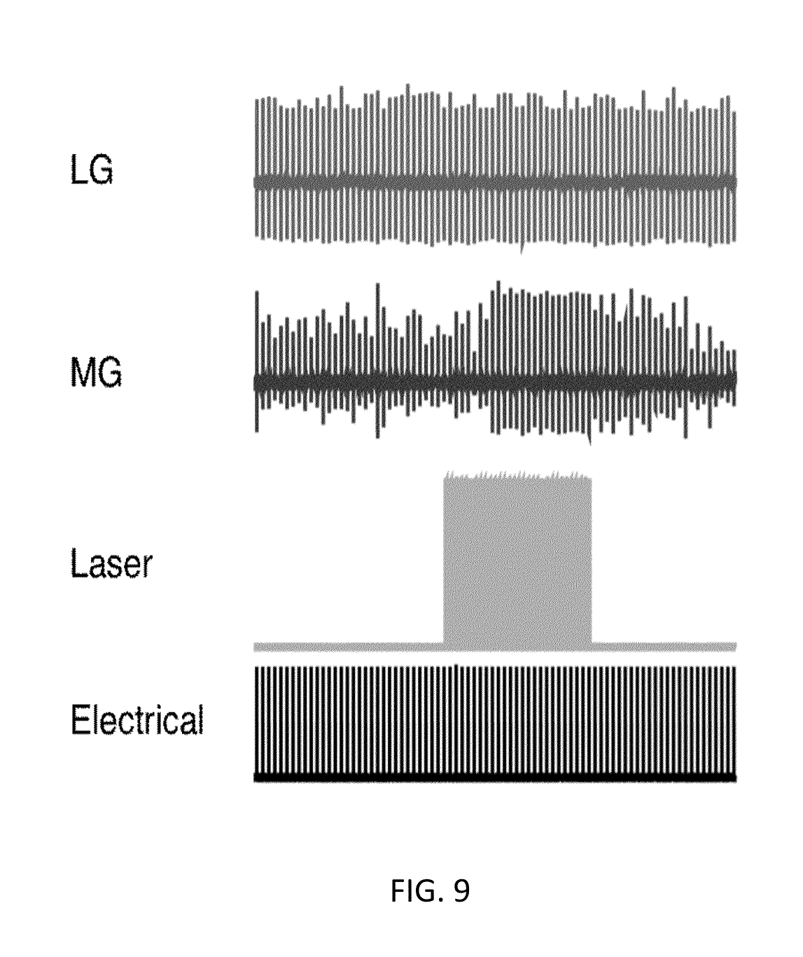

Infrared inhibition of propagating action potentials was also demonstrated in a myelinated mammalian nerve. Applying infrared pulses to the tibial branch of the rat sciatic nerve, distal to the site of electrical stimulation, reduced evoked EMG amplitude of the lateral gastrocnemius (LG) (FIG. 4) or the medial gastrocnemius (MG). Preliminary results indicate that infrared pulses are also capable of enhancing the propagating response, depending on which elements of the motor pool are recruited electrically (FIG. 9).

Both inhibition and enhancement of propagated responses were observed in the rat sciatic nerve. Whether inhibition or enhancement occurred depended on the location of the fiber optic relative to the nerve and the portion of the motor pool recruited electrically. By moving the fiber optic to different locations on the nerve we were able to see both inhibition and enhancement, though inhibition occurred the majority of the time. By changing the relative locations of the stimulating and return electrodes we were able to evoke different EMG responses, which correlated to either inhibition or enhancement.

As disclosed above, the results presented in this example demonstrate that infrared light can be used as a non-contact, artifact free and highly reversible form of precise neural inhibition. This technology is conducive to miniaturization for the control of single neurons as well as implementation into a multi-site array for governing larger neuronal populations. Pulsed infrared light is known to achieve spatially and temporally precise neural stimulation [21, 22]. Combining infrared stimulation with infrared inhibition offers the potential for full and precise control of a neural system with a single modality.

The ability to selectively inhibit the initiation and/or propagation of neural activity may have significant implications for neural prostheses and therapies. Primary challenges facing electrical neural prostheses are fractionation of spatial recruitment and mirroring of the physiological recruitment order (i.e., smaller diameter fibers before larger diameter fibers). By inhibiting the generation of selected electrically evoked responses as shown in FIG. 1, overall selectivity of electrical neural prostheses could be enhanced without sacrificing robust and reliable stimulation characteristic of electrical techniques. The results of the example also indicate the potential to inhibit neurons by size. Branch a of BN2 is composed primarily of sensory neurons, which are typically of smaller diameter than motor neurons, and was largely uninhibited during our investigation. For BN2b and BN2c, the generation of larger action potentials corresponding to motor neurons was often inhibited first, though increasing the infrared radiant exposure inhibited generation of most or all units on a given branch. By preferentially inhibiting the generation of large motor units, electrical stimulation could be steered to follow the physiological recruitment order of small fibers before large fibers. This would reduce muscle fatigue, as was recently demonstrated using optogenetic techniques [23].

The use of infrared light addresses potential limitations of the current alternatives to the block of neural propagation. The use of high frequency alternating current (HFAC) is an electrical method for blocking the propagation of neural potentials that is nearing clinical implementation. However, a challenge to this approach is the electrically evoked activity that occurs at the onset of the blockade [24]. Here we demonstrate selective inhibition of propagating action potentials without inducing increased activity at any point during the block. Rapid nerve cooling is fast acting, reversible and lacks any onset activation, but this technique will be difficult to miniaturize and is unlikely to match the spatial specificity of a laser-based approach [25]. Furthermore, the telecommunications and computing industries are driving the development of advanced laser technologies, and spatially-precise miniaturized implantable laser sources are being developed for infrared stimulation applications [26, 27]. Optogenetic methods have become increasingly popular due to the ability to selectively excite and silence neurons with spatial and temporal precision [2], but these approaches require genetic manipulations that are currently confined to limited species and non-clinical uses [3]. In contrast, pulsed infrared light is also capable of spatial and temporally precise excitation and inhibition, but without the need for viral vectors or transgenic species.

As infrared inhibition is a thermally mediated phenomenon, the ultimate application of this technique will be contingent on the absence of thermally induced changes in tissue morphology or function. While infrared radiant exposures required to inhibit action potential generation are much lower than stimulation thresholds reported previously [14], the local temperature rise required for propagation block in Aplysia is approximately 8° C. (FIG. 6). This is much lower than recent theoretical modeling predicted [17]. In the Aplysia experiments reported here, which were done at room temperature (about 20° C.), responses were stable and no functional deficits (e.g., change in neural response or evoked force) were observed across multiple nerves during hours of intermittent stimulation. Visually identifiable thermal damage was not observed, though low temperature thermal damage is not always possible to detect visually or with traditional light microscopic techniques [28]. Thermal injury is dependent on laser power (i.e., temperature) and the duration for which the temperature is maintained. Thermal damage is reversible if the duration is sufficiently short, with low temperature damage reversible for exposures ranging from 25 min to several hours [28, 29]. Infrared radiant exposures required for neural stimulation are reduced by combining the infrared stimulus with a sub-threshold electrical stimulus [13, 14]. A similar strategy may be employed to minimize the requisite temperature for potential applications of infrared inhibition.

Mou et al. proposed that thermal block of action potential propagation would require greater temperature increases than for inhibition of action potential initiation [17]. This is likely due to the action potential safety factor, which allows propagation to continue even when local excitability is reduced [30]. The excess current available for action potential propagation may explain why we experienced more robust block of action potential initiation than propagation. As Mou et al. showed, either a greater increase in temperature for one node or a lesser temperature rise distributed over multiple nodes may be required to block propagation.

Thermal neural inhibition using infrared light provides a simple tool for neural control that will aid both neural circuit analysis and the development of therapies for treating neurological disorders. Because of its simplicity, it is likely that there will be widespread and diverse application of this technique across a wide array of species and preparations.

### Example Two

### Spatial and Temporal Variability in Response to Hybrid Electro-Optical Stimulation

Hybrid electro-optical neural stimulation is a novel paradigm combining the advantages of optical and electrical stimulation techniques while reducing their respective limitations. However, in order to fulfill its promise, this technique requires reduced variability and improved reproducibility.

Among other things, one of the objectives of this example was to identify common factors that play a role in and may be controlled to enhance the reproducibility of hybrid electro-optical stimulation. Using this methodology, relevant sources of variability were identified in an experimentally tractable and relatively simple neurobiological system. These variability sources were tested in a more clinically relevant model, where the complexity of the neural system may obscure their detections. Accordingly, the experimental procedures differ slightly between the two model neural systems. However, the purpose of this example is to analyze and assess the overarching trends rather than the minor differences in stimulation protocols. To accomplish these goals, the choices of neural systems are the buccal nerve of the invertebrate marine mollusk Aplysia californica and the sciatic nerve of the vertebrate mammal Rattus norvegicus (rat). The Aplysia buccal ganglion provides a tractable, robust nervous system with large identified neurons and relatively few axons per nerve [50, 51]. These advantages facilitate the systematic empirical exploration of potential factors underlying the reproducibility of hybrid stimulation. The myelinated rat sciatic nerve is a more clinically relevant model for hybrid stimulation, but it is less robust than Aplysia nerves, and the fundamental interaction between the optical and electrical stimuli is confounded by the presence of myelin and a less stable nerve preparation. Therefore, the example identifies and characterizes factors contributing to the reproducibility of hybrid stimulation in the Aplysia buccal nerve and then evaluates those factors in the rat sciatic nerve to determine whether similar trends are observed. In this exemplary study, both spatial and temporal factors that may be controlled to reduce variability and enhance reproducibility were investigated.

There are two aspects of the spatial component that are addressed: (1) the relative locations of the optical and electrical stimuli and (2) the size of the excitable region as a function of the optical stimulus strength. The mechanism of INS was shown to involve a thermal gradient [52]. Thus, it is assumed that the thermal gradient and the electrical current path must overlap spatially. However, what is not known is where this overlap may occur, or how the two fields may affect each other. The activating function, which describes the transmembrane potentials leading to the electrical activation of a neuron, results in neurons closest to the cathode being activated first (with larger axons recruited before smaller axons) [53, 54]. Experimentally, stimulation threshold current is shown to increase with increasing distance from the cathode [55]. Given that the electrical stimulus preferentially targets neurons nearest the cathode, it is hypothesized that hybrid stimulation requires the lowest optical pulse energies when the optical stimulus is located along the electrical current path and adjacent to the cathode. Like electrical stimulation, increasing INS radiant exposures results in an increase in magnitude of the evoked response, suggesting recruitment of additional axons [56]. Therefore, it is expected that for a given sub-threshold electrical stimulus, an increase in the sub-threshold optical stimulus yields an increase in the size of the excitable region for hybrid stimulation.

It has long been known that electrical stimulation thresholds vary over time [57]. In examining temporal factors, it is evaluated how brief fluctuations (minutes) and long-term trends (minutes to hours) in electrical stimulation thresholds affect optical pulse energies for hybrid stimulation. Correct measures of optical energies for hybrid stimulation require an accurate determination of the electrical ‘priming’ stimulus at the time of the measurement. If one incorrectly assumes that the electrical stimulation threshold is stationary over a fixed period of time, then hybrid stimulation performance will suffer. To address this issue, threshold optical energies for hybrid stimulation is measured while monitoring electrical thresholds over an extended period of time. It is hypothesized that if the electrical threshold is known at any point in time, then the additional optical energy required for stimulation can be predicted for a given sub-threshold stimulus. Additionally, changes in threshold radiant exposures for the optical component of hybrid stimulation are positively correlated with the changes in the underlying electrical stimulation threshold.

In this example, a comparative physiological approach was employed to aid the further development of this technique by identifying the spatial and temporal factors characteristic of hybrid stimulation that contributes to experimental variability and/or a lack of reproducibility. Using transient pulses of infrared light delivered simultaneously with a bipolar electrical stimulus in either the marine mollusk Aplysia californica buccal nerve or the rat sciatic nerve, the existence of a finite region of excitability with size altered by the strength of the optical stimulus and recruitment dictated by the polarity of the electrical stimulus was determined. Hybrid stimulation radiant exposures yielding 50% probability of firing (RE₅₀) were shown to be negatively correlated with the underlying changes in electrical stimulation threshold over time. In Aplysia, but not in the rat sciatic nerve, increasing optical radiant exposures (J/cm²) beyond the RE₅₀ ultimately resulted in inhibition of evoked potentials. Accounting for the sources of variability identified in this study increased the reproducibility of stimulation from 35% to 93% in Aplysia and 23% to 76% in the rat with reduced variability.

### Materials and Methods

### Aplysia Californica Preparation and Electrophysiology

Aplysia californica (n=26) weighing 190-250 g (Marinus Scientific, Newport Beach, Calif.) were maintained in an aerated aquarium containing circulating artificial seawater (ASW) (Instant Ocean; Aquarium Systems, Mentor, Ohio) kept at 16-17° C. The animals were fed dried seaweed every 1-3 days.

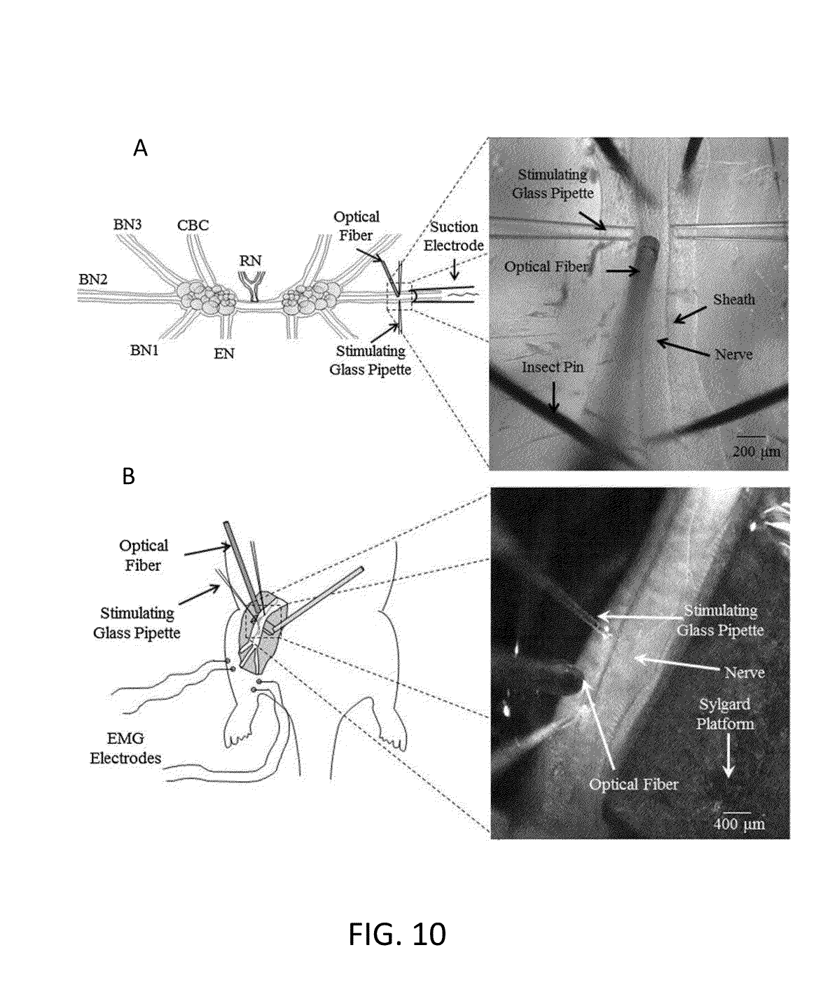

Aplysia were anesthetized with an injection of 333 mM MgCl2 (50% of body weight) prior to dissection. Once anesthetized, animals were dissected and the buccal ganglia were removed and pinned in a recording dish and immersed in Aplysia saline (460 mM NaCl, 10 mM KCl, 22 mM MgCl2, 33 mM MgSO4, 10 mM CaCl2, 10 mM glucose, 10 mM HEPES, pH 7.6). Once dissected and pinned, Aplysia nerves were left untreated so as not to reduce spontaneous activity. We chose not to discard data from trials where spontaneous activity occurred, as excitability varies with the level of activity. This is an inherent biological factor to be assessed in the exemplary study. For each experiment, the nerve of interest (either buccal nerve 2 (BN2) or buccal nerve 3 (BN3)) was anchored in place by pinning the protective sheath around the nerve to the Sylgard base (Dow Corning, Midland, Mich.) of the recording dish. Once securely pinned, the nerve to be investigated was suctioned into a nerve-recording electrode to monitor the response to stimulation (FIG. 10A). Nerve suction recording electrodes were made by hand-pulling polyethylene tubing (1.27 mm outer diameter; PE90; Becton Dickinson) over a flame to the desired thickness. Recording electrodes were suction-filled with Aplysia saline prior to suctioning of the nerve. Nerve signals were amplified (×1000) and band-pass filtered (300-500 Hz) using an ac-coupled differential amplifier (model 1700; A-M Systems), digitized (Axon Digidata 1440A; Molecular Devices, Sunnyvale, Calif.) and recorded (Axograph X; Axograph Scientific).

### Rat Preparation and Electrophysiology

All rat experiments were performed following protocols approved by the Institutional Animal Care and Use Committee. Female Sprague-Dawley rats (n=9) weighing 150-200 g (Charles River) were anesthetized with continuously inhaled isoflurane (induction: 3% isoflurane, 2.0 LPM oxygen; maintenance: 2-2.5% isoflurane, 1.5 LPM oxygen). A rectal probe and heating pad (catalog 40-90-8, FHC, Bowdoin, Me.) were used to maintain the rat at a target body temperature of 35-37° C. throughout the experiment. The lateral sides of the animals' back legs were shaved and the sciatic nerve exposed proximal to the knee via an incision in the overlying muscle. The muscular fascia over the nerve was removed while the nerve's epineurial layer was left intact. Saline was added periodically to keep the nerve from dehydrating throughout the experiment. A custom Sylgard platform was anchored to a micromanipulator and placed below the sciatic nerve with minimal added tension to minimize motion of the nerve due to the animal's respiration (FIG. 10B). Evoked muscle action potentials were recorded using paired needle electrodes inserted in the areas of the biceps femoris and gastrocnemius muscles. EMG signals were amplified (×1000), band-pass filtered (300-1000 Hz), digitized and acquired using the same setup as for Aplysia.

### Endpoint Definition

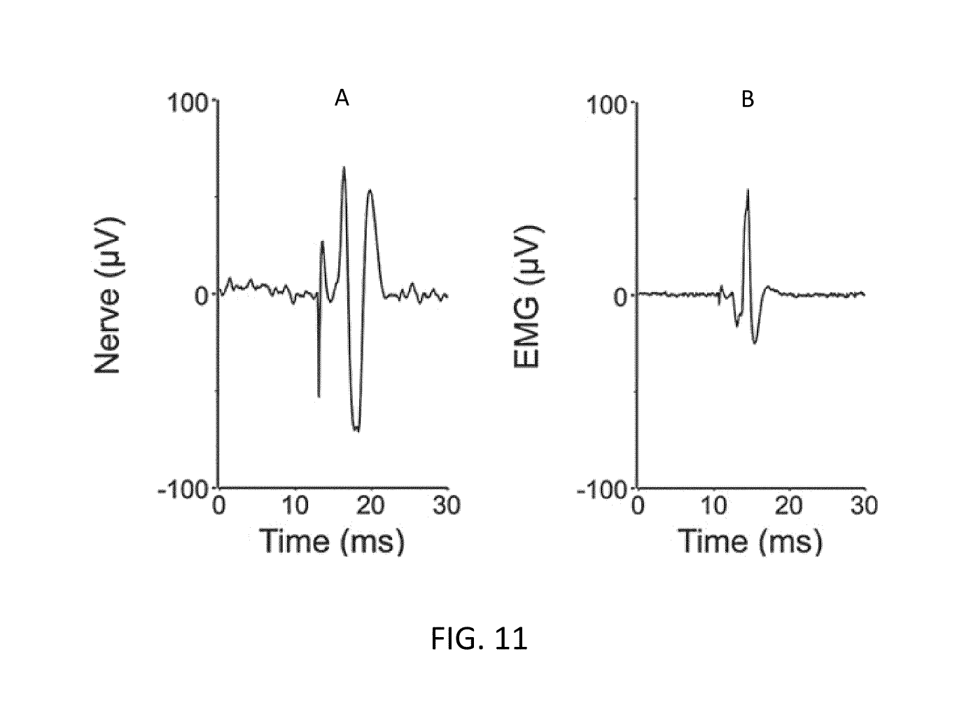

Analysis of hybrid stimulation requires an appropriately defined endpoint. In Aplysia, the endpoint is defined as the visible detection of single and/or compound extracellular nerve spikes in response to stimulation (FIG. 11A). Similarly, the endpoint for the rat experiments was visibly identified single and/or compound muscle action potentials in response to stimulation (FIG. 11B). For both species, we also required that the evoked potentials were frequency locked with the repeating stimulus (i.e., constant delay following a presented stimulus pulse) to distinguish evoked responses from spontaneous activity.

### Electrical and Optical Stimulation

Extracellular stimulating electrodes were made from thin-wall borosilicate capillary glass (catalogue 615000; A-M Systems, Everett, Wash.) pulled to resistances of about 0.2 MQ (PP-830; Narishige). For each Aplysia experiment, two electrodes were capillary filled with Aplysia saline and placed on either side of the nerve in contact with the nerve sheath. This created a bipolar stimulus, with the pipettes oriented transverse to the longitudinal axis of the nerve. Pipettes were positioned such that their angle of approach to the nerve was as shallow as was allowed by the edge of the recording dish. For the rat experiments, two glass pipettes were filled with normal saline and placed in contact with the nerve along the nerve's longitudinal axis. The stimulating pipette arrangement for each species was chosen based on consistency of stimulation thresholds and ability to achieve reliable supra-threshold stimulation on each nerve tested. Monophasic currents were supplied by a bipolar stimulus isolator (A365R; WPI) and passed between the two pipettes in each preparation. Electrical stimulation was defined as the minimal current that would yield five consecutive evoked potentials in response to pulsed stimuli.

For the optical stimulation, both a holmium:yttrium-aluminum-garnet (Ho:YAG) solid state laser (SEO Laser 1-2-3, Schwartz Electro-Optics, Orlando, Fla.) and a tunable pulsed diode laser were used (Capella; Lockheed-Martin-Aculight, Bothwell, Wash.). Two different lasers were chosen due to the established performance in peripheral nerves offered by the Ho:YAG and the ease of use and INS-specific design of the Capella. While the Capella was used in our previous demonstration of hybrid nerve stimulation, the Ho:YAG is the laser of choice for much of the INS literature pertaining to peripheral mammalian nerves [46-48, 52, 58, 59]. However, the Capella offers vastly improved ease of use and greatly reduced pulse-to-pulse variability when compared with the Ho:YAG. The Capella is also known to work exceptionally well for INS in a wide array of excitable tissues including the cochlea, somatosensory cortex, embryonic heart, cardiomyocytes and the vestibular system [60-64]. While the Ho:YAG provides pulses of infrared light (λ=2.12 μm) having fixed pulse duration (τp=0.25 ms), the Capella has slightly tunable wavelength (λ=1.855-1.875 μm) and a variable pulse duration. The important parameter for INS is penetration depth in tissue (as pulse duration was shown to have negligible effects [17]); therefore, the Capella is set to have a wavelength of λ=1.875 μm for all experiments to match the absorption (i.e., penetration depth) of the Ho:YAG laser [65].

For the Aplysia experiments, laser output was coupled into either a flat-polished 100 or 200 μm diameter optical fiber (Ocean Optics, Dunedin, Fla.). For each experiment, the tip of the optical fiber was immersed in the Aplysia saline bath and brought into contact with the nerve sheath. The optical fiber was then slowly retracted with a micromanipulator and gently translated back and forth transverse to the nerve until the optical fiber was just out of contact with the nerve sheath. For radiant exposures presented in this study, the laser-irradiated area is assumed to be a circular spot on the incident surface of the nerve sheath having diameter equal to that of the optical fiber (i.e., 0.0314 mm² for a 200 μm fiber and 0.00785 mm2 for a 100 μm fiber). For simplicity, as the optical fiber is just out of contact with the nerve sheath, this assumes no divergence of the beam from the tip of the optical fiber to incident surface of the nerve sheath.

For the rat experiments, laser output was coupled into a flat-polished 400 μm diameter optical fiber (Ocean Optics, Dunedin, Fla.). The fiber diameter for rat experiments was chosen to match the 400-600 μm optical fibers used in mammalian peripheral nerve studies, while smaller fibers were used in Aplysia studies to scale with the size of the Aplysia buccal nerves [49, 58, 59]. The optical fiber was positioned 500 μm from the incident surface of the nerve at an angle just off of vertical with a layer of saline just covering the surface of the nerve. The laser-spot size was measured using the knife-edge technique where two perpendicular measurements were taken along the axes of the presumed circularly shaped laser spot, yielding an irradiated area of 0.19 mm² [66]. Pyroelectric energy detectors were used to measure pulse energies from the tip of the optical fiber for the Ho:YAG laser (J25, Coherent-Molectron Inc., Santa Clara, Calif.) and Capella laser (PE50BB-SH-V2, Ophir Optronics Ltd).

For INS alone, an optical stimulation threshold was defined as the minimum radiant exposure that would yield five consecutive evoked potentials in response to pulsed stimuli. In the Aplysia buccal nerve, using the Capella laser coupled to a 200 μm optical fiber that was retracted just out of contact with the nerve, threshold radiant exposures averaged 8.93 J/cm² with a 95% confidence interval of 8.72-9.14 J/cm² (25 measurements from 7 nerves). In the rat sciatic nerve, using the Ho:YAG laser coupled to a 400 μm optical fiber, threshold radiant exposures averaged 1.12 J/cm² with a 95% confidence interval of 0.92-1.32 J/cm² (12 measurements from 8 nerves).

Previous published studies found threshold radiant exposures in mammalian peripheral nerves ranging from 0.32 to 1.77 J/cm² [46-49, 52, 58, 59]. However, directly comparing these values with published data is difficult. Ongoing studies in our lab show stimulation thresholds in the rat sciatic nerve from 0.7 to 1.3 J/cm² (unpublished). In the cochlea, stimulation thresholds are on the order of mJ/cm² [67]. To make direct comparisons, it is imperative that certain factors be controlled; in particular, spot-size determination and measures of threshold must be the same. Radiant exposures are highly dependent on the spot-size. Differences in the way spot-sizes are calculated or measured between studies propagate into large differences in reported radiant exposures (due to the squared term in the denominator). In addition to variations in experimental preparations (i.e., neural model system, in vivo, ex vivo or in situ), thresholds may vary based on the definition of the endpoint for a given study, for example, whether the threshold is defined by the appearance of muscle or nerve action potentials, or by a visibly identified muscle twitch [47, 49, 67]. A noteworthy aspect of this study is that no visible damage or loss of function (as indicated by the response to electrical stimulation) was noted as a result of stimulation with the radiant exposures used. This is particularly relevant to Aplysia, where optical- and hybrid-evoked potentials remained steady over several hours of stimulation (not shown).

All nerve stimulation was coordinated through computer software (AxoGraph X; AxoGraph Scientific, Sydney, Australia) and applied at a repetition rate of 2 Hz. In both preparations, electrical pulses of 100 μs were used. Optical pulse durations were 250 μs for the Ho:YAG and 2-3 ms for the Capella lasers, respectively. This is due to the fixed pulse duration of the Ho:YAG and the minimum pulse duration of the Capella required to achieve optical energies for stimulation. Since the underlying mechanism of INS has been shown to be thermally mediated and dependent on a temperature gradient [52], as long as the pulse duration is significantly shorter than the thermal diffusion time (about 100 ms), the laser pulse can be considered as an input delta function to the system. For hybrid stimulation, pulses were synchronized such that they ended concurrently. This allowed for the total charge and total thermal deposition to occur simultaneously. Nerve recordings were triggered and acquired for 10 ms prior to stimulation through 140 ms post stimulation.

### Experimental Methods for Spatial Factors

To investigate spatial factors contributing to the reproducibility of hybrid stimulation, sub-threshold pulses of electrical current (90% of electrical stimulation threshold) were applied simultaneously with optical pulses of a set magnitude. During hybrid stimulation, the optical fiber was translated across the nerve between the stimulating pipettes using a micromanipulator. A CMOS color USB camera and accompanying software (catalog 59-367; Edmund Optics, Barrington, N.J.) were used to record the position of the optical fiber. A LED was triggered by computer software to flash synchronously with the laser pulse so that we could reconstruct the exact position of the optical fiber at the time of stimulation. The center of the tip of the optical fiber was plotted and correlated with the presence or absence of stimulation as indicated by an evoked potential on the nerve recording.

### Experimental Methods for Temporal Factors

Temporal factors were examined by investigating how fluctuations in the electrical stimulation threshold over time affect the optical component of hybrid stimulation. Threshold currents were measured every 2-3 min for 1-3 hr to monitor underlying changes in electrical stimulation with time and to assure that hybrid stimulation was not inducing alterations in threshold currents. One hour of each trial was an experimental period where radiant exposures eliciting hybrid stimulation were measured along with electrical stimulation threshold currents. Every 2-3 min during this experimental period, electrical stimulation threshold currents were first measured and then the stimulus current was reduced to 90% of electrical stimulation threshold. For the Aplysia experiments, five pulses of five different radiant exposures were then systematically applied with the sub-threshold current pulses. For the rat experiments, eight pulses of five different radiant exposures were applied. The order in which the radiant exposures were applied was determined by a random sequence generator so as to limit any conditioning effects or bias. Each hybrid stimulus pulse was recorded as either a 1 or 0 as determined by the presence (1) or absence (0) of a visibly identified nerve (Aplysia) or muscle (rat) action potential. This process was repeated every 2-3 min for the duration of the experimental period.

### Data Analysis

For spatial data, movie files were analyzed with custom software (Matlab r2010b; Mathworks, Natick, Mass.). Locations of successful stimulation were compared using non-parametric statistical tests. The two-sample Kolmogorov-Smirnov test compares two empirical distributions and responds to both the overall shape and location of the distributions. While this test indicates if the distributions are statistically different, it does not tell whether it is due to the relative size or location of the distributions. To distinguish whether differences are due to changes in size or location of the region of excitability (ROE), the Mann-Whitney test was also performed, which is a non-parametric test that determines if the median of one data set is greater than another. The interquartile range was used as a measure of the size of the ROE.

Temporal data were aggregated using Matlab with statistical analysis performed in Microsoft Excel (Microsoft Office Professional Plus 2010) and Slide Write Plus Version 6 (Advanced Graphics Software, Inc., Encinitas, Calif.). For each radiant exposure, the number of ones was divided by the sum of ones and zeros to achieve a probability of firing. The cumulative distribution function (CDF) of the standard normal distribution,

$$F ( x : μ , {σ}^{2} ) = \frac{1}{2} [ 1 + erf ( \frac{x - μ}{σ \sqrt{2}} ) ] , x ∈ , \qquad ( 1 )$$

where x is a random variable with mean μ and variance σ², was then fitted to the data to determine the radiant exposure yielding 50% probability of firing (RE₅₀). While the RE₅₀ is not practically useful for stimulation, we use this approach as a generally well-accepted model for making comparisons and identifying thresholds [64, 68-71]. One of the objectives of the invention is to establish a methodology and identify pertinent considerations for successful hybrid stimulation rather than prescribe optimal conditions for stimulation.

### Results and Discussions

### Existence of a Bounded Excitable Region

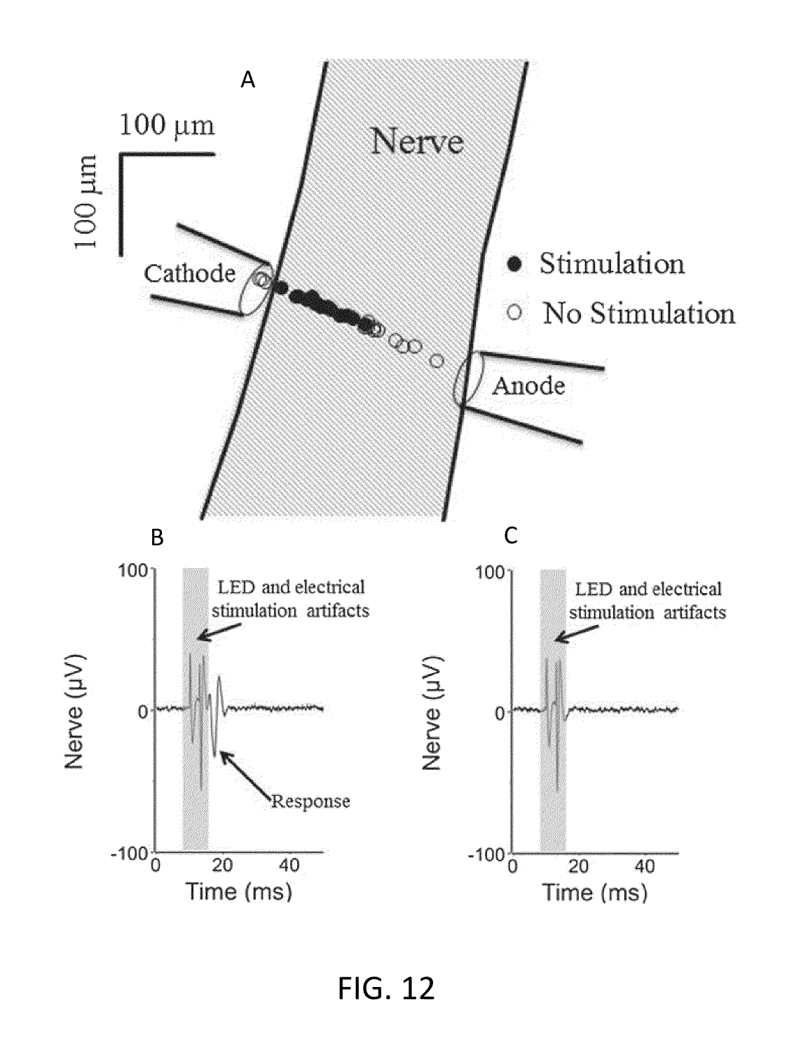

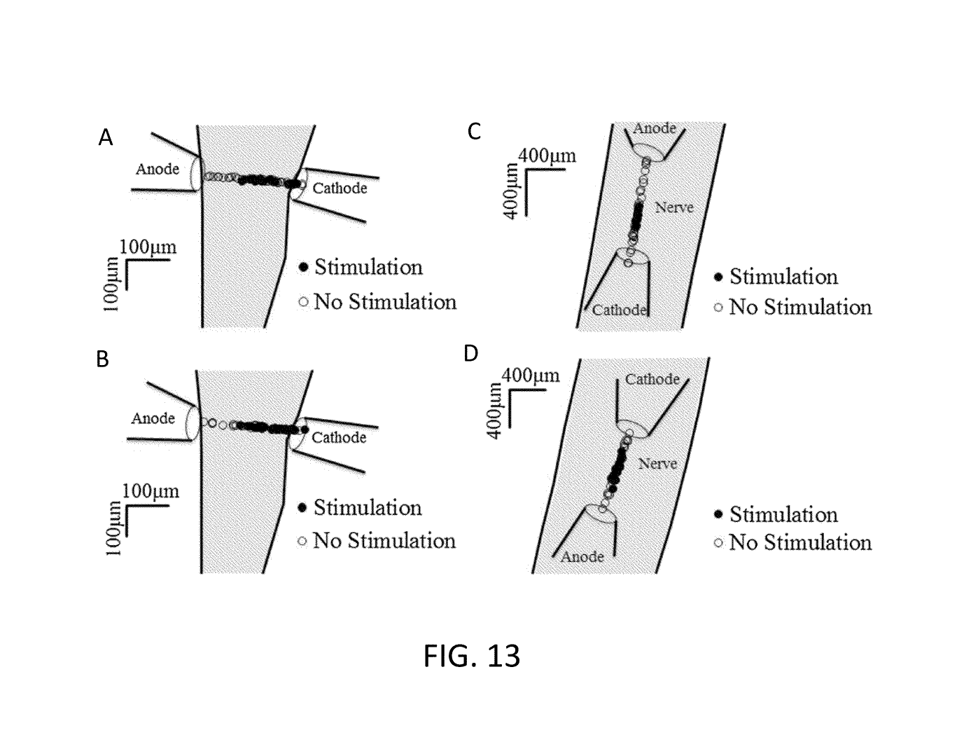

When translating the optical fiber back and forth across the nerve, it was determined that there exists a finite region between the cathode and anode where hybrid stimulation is possible (FIG. 12). This was observed in all of the nerves tested for both Aplysia (n=42) and the rat (n=13). However, in two rat sciatic nerves, some experimental trials yielded locations of successful hybrid stimulation extending outside of this finite region. During these trials, the electrical stimulation threshold was more variable. Occasionally, the electrical component of hybrid stimulation approached electrical stimulation threshold, raising the overall excitability of the nerve. For both Aplysia and the rat, there were variations in the size and shape of evoked responses between animals, nerves and locations within a single nerve. This suggests that multiple different axons were recruited over the course of the experiments. In each species, there were ROEs including only a single evoked unit and others that exhibited different units depending on the location of the optical fiber and the intensity of the optical stimulus. No apparent differences in ROE were observed when comparing the Capella and Ho:YAG within a single nerve (FIGS. 13A and 13B) or across animals (FIGS. 13C and 13D) for Aplysia or the rat. However, the yield with the Ho:YAG in the rat sciatic nerve was greater due to more reliable optical stimulation. With no obvious differences between the lasers other than overall yield, greater emphasis was placed on the Capella for the remaining Aplysia experiments (due to its ease of use and consistent pulse energies) and the Ho:YAG for the rat (due to the superior results it provided for myelinated nerve fibers).

### Size of the ROE

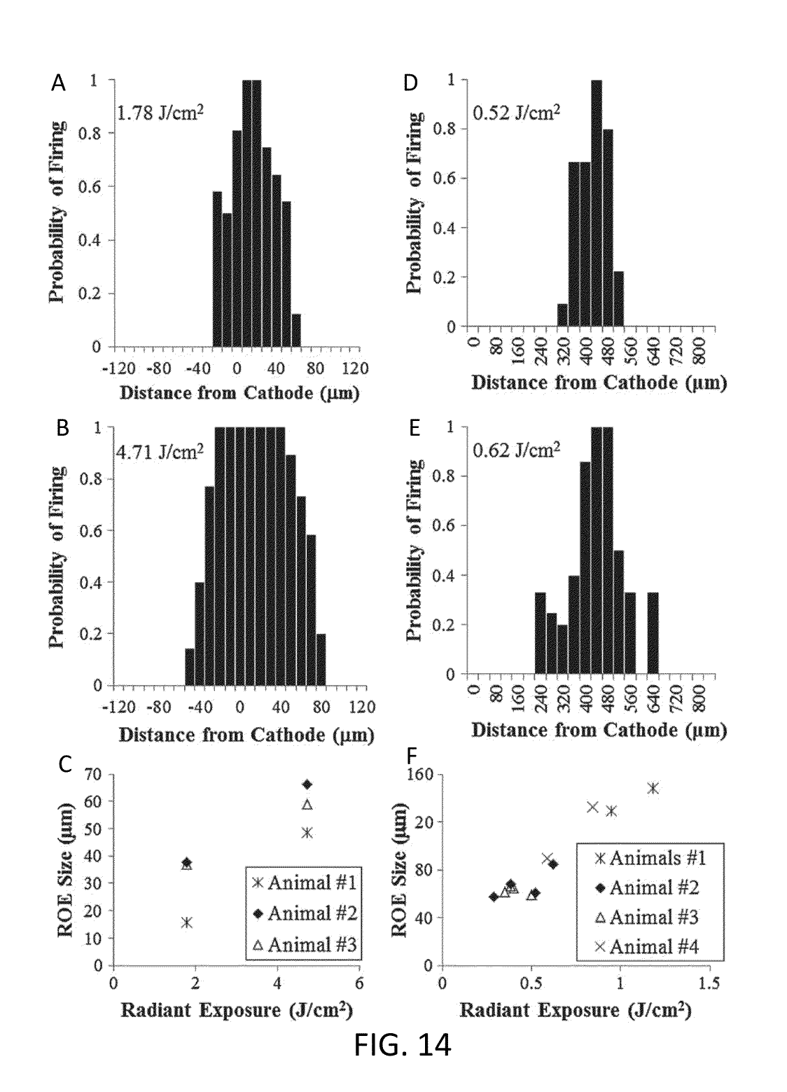

After identifying the existence of a finite ROE, how the strength of the optical stimulus altered its size was investigated. With electrical current at 90% of electrical stimulation threshold, the ROE size for optical stimuli of 1.78 and 4.71 J/cm² using the Capella in Aplysia and 0.29-1.18 J/cm² was compared with both the Ho:YAG and Capella lasers in the rat. These values were chosen to cover a range of optical radiant exposures that, in the absence of the electrical stimulus, are sub-threshold for stimulation in their respective neural systems. Locations of hybrid stimulation were binned and plotted as a probability histogram by dividing the number of stimuli evoking a response by the total number of attempts for each bin (FIGS. 14A, 14B, 14D and 14E). After confirming that the ROE median was the same for each radiant exposure (using the Mann-Whitney test), the two-sample Kolmogorov-Smirnov test was applied to determine if the sizes of the distributions were significantly different.

In Aplysia, a total of 28 trials were acquired from 3 nerves (3 different animals). In the rat, a total of 26 trials were acquired from 4 nerves (4 different animals). Equal radiant exposures from the same nerve and animal were combined into one data set. In Aplysia, a statistically significant increase (p<0.05) in the ROE size with increasing radiant exposure was observed for all nerve tested (FIG. 14C). For the rat, the results indicated a statistically significant increase in the ROE size (p<0.05) for one of the four animals tested (FIG. 14F) and an insignificant increase (p>0.05) for the remaining nerves. However, combining the results from all four rat nerves shows a linear increase in ROE size across the radiant exposures tested. The lack of statistical significance in three of the four rat nerves tested is likely due to the limited range of radiant exposures tested in each nerve. However, the center of each ROE showed a greater probability of firing at the higher radiant exposure in all nerves (not shown).

### Effects of Stimulus Polarity

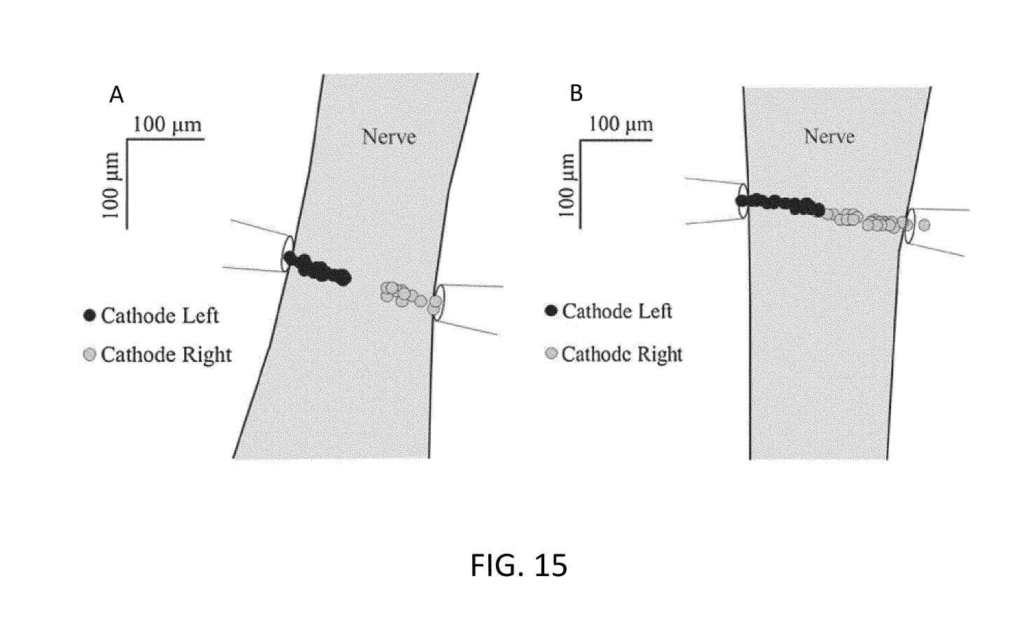

It was hypothesized that the polarity of the electrical stimulus would shift the location of the ROE. To test this, the ROE was identified as before, and then the polarity was reversed (while keeping the electrodes in place) and the new ROE was found. In Aplysia, this experiment was repeated using both the Capella and Ho:YAG lasers with a constant optical stimulus (2.42-4.71 J/cm²) across a total of 8 nerves from 7 animals yielding 11 polarity pairs. The Mann-Whitney test was used to evaluate whether a shift in the ROE median occurred with a change in polarity. For all polarity pairs, a reversal in polarity showed a statistically significant shift (p<0.05) in the ROE median such that the ROE was located adjacent to the cathode (FIG. 15). This demonstrates that, for a given electrode arrangement, two unique ROEs may be achieved by simply reversing the direction of the current path. In the rat sciatic nerve, effects of polarity were investigated using both the Ho:YAG and Capella lasers in a total of six nerves from four animals. A statistically significant shift in the ROE median was observed in three of the six nerves tested. Of the three nerves not showing a statistically significant shift in the ROE median, two exhibited successful hybrid stimulation with only one polarity. While statistically significant shifts in the ROE median were observed in half of the nerves tested, changes in location were not as dramatic as in the Aplysia.

### Effects of Electrical Stimulation Threshold on Hybrid Stimulation

Electrical stimulation threshold currents as well as the RE₅₀ for hybrid stimulation were monitored in the same nerve to determine if fluctuations in the former affect the latter. The RE₅₀ for hybrid stimulation was determined by first generating probabilities of firing at a given radiant exposure for each time point (by dividing the number of stimulation attempts evoking a response by the number of total attempts) and then fitting those probabilities to a CDF (Equation (1)). The RE₅₀ was defined as the radiant exposure providing a 50% probability of firing as indicated by the CDF fit.

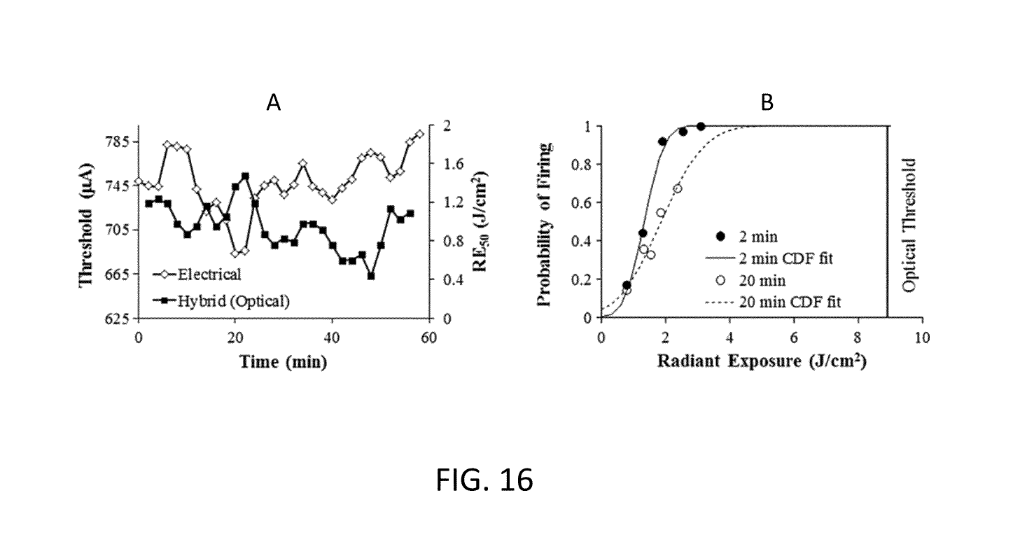

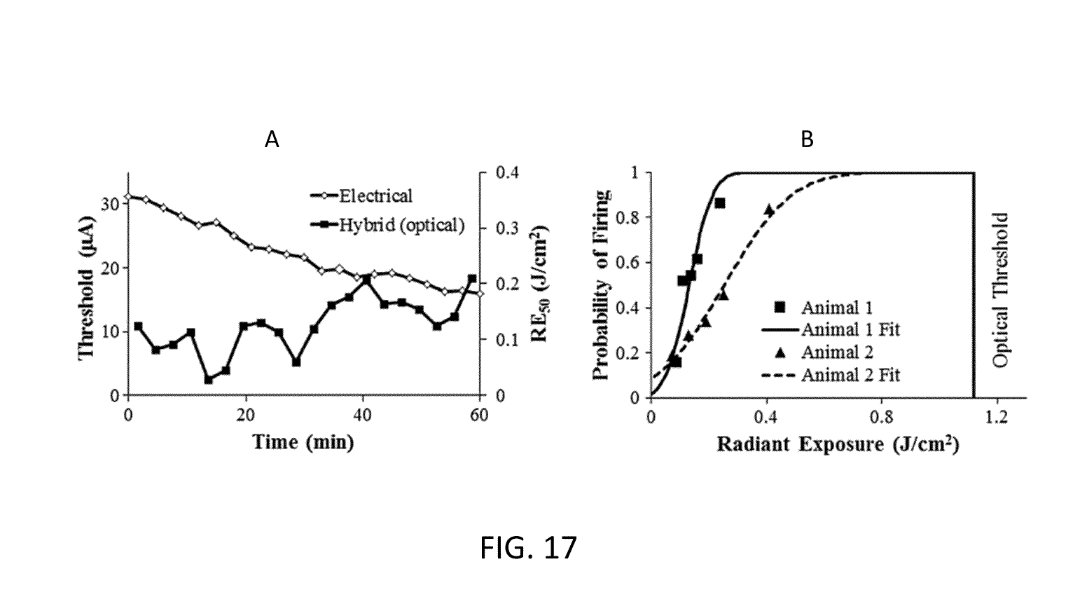

For the Aplysia, 5 pulses of 5 radiant exposures (using the Capella laser) yielded 25 total data points every 2 min. These data were not sufficient for a reliable CDF fit at each time point, so a sliding window was applied to fit a CDF to 6 min windows of data. FIG. 16A provides an example of the changes in thresholds for electrical stimulation and the optical component of hybrid stimulation over an hour. Each of the four Aplysia buccal nerves tested had a statistically significant (p<0.05) negative correlation between thresholds for electrical stimulation and the optical component of hybrid stimulation. In the rat, 8 pulses of 5 radiant exposures (using the Ho:YAG laser) yielded 40 total data points every 3 min. A sliding window was applied to fit a CDF to 6 min windows of data. Of the two nerves tested, one exhibited a statistically significant (p<0.05) negative correlation between thresholds for electrical stimulation (FIG. 17A) and the optical component of hybrid stimulation and the other showed an insignificant (p>0.05) negative correlation.

To evaluate the consistency over time of the RE₅₀ for hybrid stimulation, all of the data acquired from a given nerve were compiled and each radiant exposure was converted to a probability of firing. The probability of firing as a function of radiant exposure was then fit to a CDF. In Aplysia, a total of four nerves from four animals (n=610 data points at each radiant exposure) yielded a 50% probability of firing at 1.34 J/cm² with a 95% confidence interval between 1.13 and 1.55 J/cm² (FIG. 16B). Here, the confidence interval is indicative of variability in hybrid stimulation RE₅₀ over the hour of measurements, where a narrow confidence interval (and increased slope of the CDF fit) indicates less variability. A subsequent set of experiments was performed in Aplysia to determine if increasing the interval between adjustments to the sub-threshold electrical stimulus yielded an increase in the confidence interval (i.e., an increase in variability). For these experiments, the electrical stimulation threshold was measured every 2 min, but the sub-threshold electrical stimulus used for hybrid stimulation was only set to 90% of electrical stimulation threshold at the 0, 20 and 40 min time points. A total of five nerves from three animals (n=610-900 data points per radiant exposure) yielded a 50% probability of firing of 1.86 J/cm² with a 95% confidence interval between 1.40 and 2.33 J/cm². When comparing the 2 and 20 min adjustment intervals, the 95% confidence interval for the 20 min adjustment is roughly twice that of the 2 min adjustment. This is also shown in FIG. 16B as a shallower slope in the probability of firing as a function of radiant exposure for the 20 min adjustment. A noteworthy aspect of FIG. 16B is that the y-intercept for the 20 min adjustment plot is greater than 0, suggesting that there is a small probability of firing even with 0 J/cm² of optical stimulus. This is due to rare occasions where the electrical stimulation threshold fell below the previously set sub-threshold stimulus before the next adjustment was made.

FIG. 17B shows the results of aggregating data from each rat for the purpose of assessing threshold radiant exposure consistency. Rather than compiling the data from both animals, each animal is plotted separately. The results indicate that threshold variability is more prominent in the rat than in Aplysia. Animal 1 has RE₅₀ of 0.13 J/cm² with a 95% confidence interval of 0.10-0.16 J/cm², whereas animal 2 has RE₅₀ of 0.25 J/cm² and a 95% confidence interval of 0.17-0.33 J/cm².

### Hybrid Inhibition

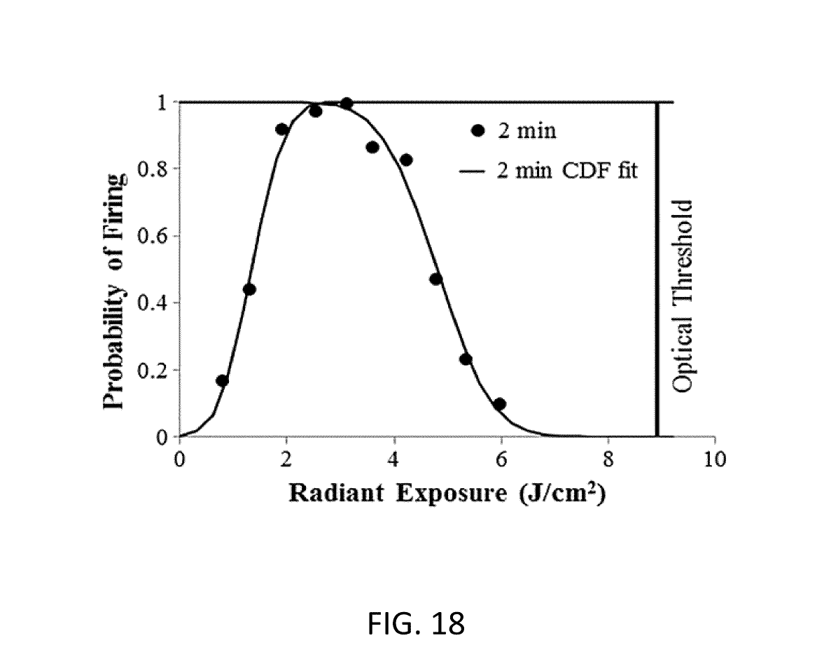

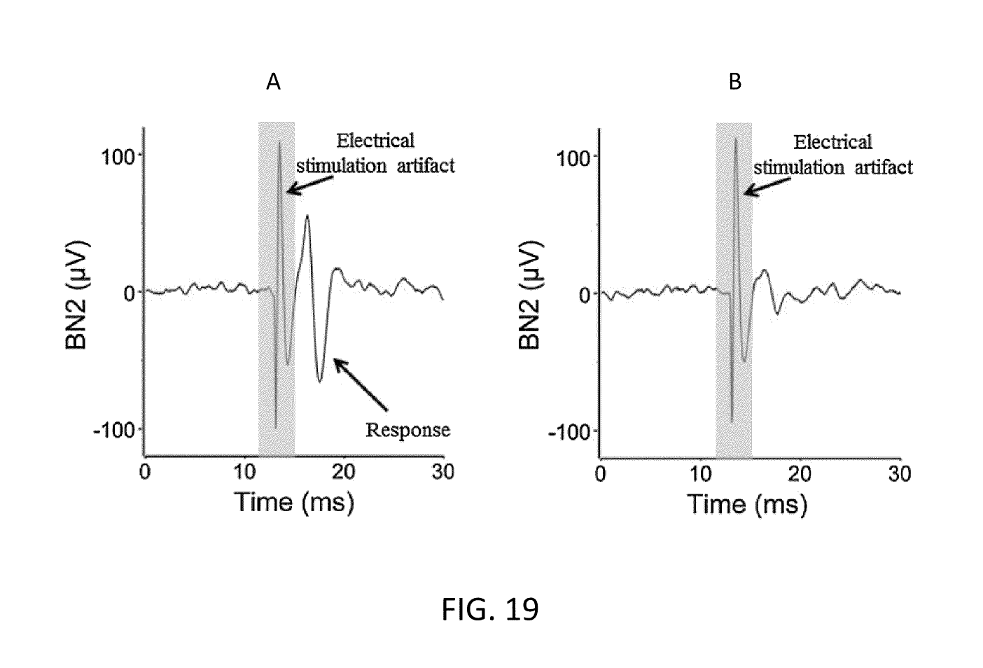

In the course of evaluating temporal factors affecting the RE₅₀ for hybrid stimulation in Aplysia, it was discovered that at higher radiant exposures, the probability of firing began to decrease rather than asymptotically approach 100% as expected. To further investigate this phenomenon, the electrical stimulus was set to 90% of electrical stimulation threshold every 2 min and five pulses of five radiant exposures were applied in the manner described above. However, for this experiment the radiant exposures were higher than those used for identifying the RE₅₀. The results from four nerves from two animals (n=600 data points per radiant exposure) are shown in FIG. 18. Interestingly, if defining stimulation as >50% probability of firing, then with an electrical priming stimulus of 90% of electrical stimulation threshold, stimulation will occur for radiant exposures from 1.34 to 4.79 J/cm² rather than >1.34 J/cm² as was initially expected. This raised the question as to whether higher radiant exposures actually inhibit neuronal firing, or whether another mechanism is activated at these radiant exposures. An electrical stimulus was applied at 110% of electrical stimulation threshold and then the optical stimulus (three nerves from three animals) was added. In each trial, the electrically evoked unit was inhibited by the optical stimulus (FIG. 19). Radiant exposures for inhibition of the electrically evoked unit averaged 7.13±0.51 J/cm² over 12 trials. It is important to note that all of these radiant exposures are below optical stimulation threshold radiant exposures and that this process is completely reversible. If radiant exposures are reduced, then the evoked response returns. Hybrid inhibition was investigated in the rat but was not observed.

Reducing the optical energy required to stimulate excitable tissues may facilitate clinical translation of infrared neural interfaces due to the reduced likelihood of thermal tissue damage, and by making the design criteria for laser sources less restrictive. The purpose of this study was to assess potential factors that might contribute to variability in hybrid electro-optical stimulation, as well as to create a methodology for reliable and reproducible hybrid stimulation. This task was approached by comparing trends seen in two different neurobiological systems—the tractable and well-characterized Aplysia californica buccal ganglion and the myelinated and more clinically relevant rat sciatic nerve. Given the variability and lack of reproducibility as previously experienced, this approach allowed for identification of factors in the more experimentally tractable system that could subsequently be applied to the more clinically relevant preparation. Some concern may arise as to the translation of hybrid stimulation between an unmyelinated, invertebrate nerve and a myelinated, mammalian nerve. However, this study shows that the information gathered from experiments in Aplysia directly led to improved understanding and performance of hybrid stimulation in the rat sciatic nerve. Although some aspects of the experimental protocol differ between the two preparations (i.e., orientation of stimulating pipettes, source of optical stimulation, endpoint definition), overarching trends were clearly evident across both species. Prior to both adopting the methods used in this study and controlling for the spatial and temporal factors we have assessed, our efficacy for hybrid stimulation in the Aplysia buccal nerve and the rat sciatic nerve was 35% and 23%, respectively (unpublished data). In this paper, we define efficacy as a nerve demonstrating a hybrid stimulation event where a sub-threshold electrical stimulus and sub-threshold optical stimulus are combined to achieve an evoked response. We attempt to determine whether or not sub-threshold electrical and optical stimuli were combined to achieve supra-threshold stimulation. At the conclusion of this study, we now have an efficacy of 93% ( 42/45 nerves) in the Aplysia buccal nerve and 76% ( 13/17 nerves) in the rat sciatic nerve.

Relative mechanical stability between the target neural tissue, optical fiber and electrodes was imperative to achieving reliable and reproducible hybrid stimulation. This allowed for consistent location of the stimuli throughout a given experiment by minimizing nerve movement due to optical fiber movement, fluid flow (Aplysia) or animal respiration (rat). Stabilization challenges are likely to be alleviated as hybrid stimulation progresses to multi-modality nerve cuff stimulators where microfabricated cuffs will be able to adapt to changes in nerve shape and movement.

The orientation of the stimulating glass pipettes is also an important part of the physical setup that must be taken into account. In the rat, electrical stimulation was more reliable with the pipettes oriented along the longitudinal axis of the nerve than in a transverse configuration. For electrical stimulation of myelinated nerves, it is necessary to induce longitudinal axonal currents, which may explain the reason that pipettes oriented longitudinally to the nerve were most effective. Recent models of intrafascicular stimulation support these observations. As a function of position relative to nodes of Ranvier, bipolar stimulation with a longitudinal configuration was shown to have less variability in threshold currents as compared to a transverse configuration [37]. While Aplysia nerves are unmyelinated, and thus do not possess nodes of Ranvier, they do exhibit clustering of voltage-gated sodium channels that may aid in the conduction of action potentials along the nerves [38]. However, it was found in Aplysia nerves that electrical stimulation was more reliable with the pipettes oriented transverse to the nerve. Due to the thick outer sheath protecting the nerve, placing the glass pipettes along the longitudinal axis of the nerve may result in electrical current dissipating into the bath rather than penetrating to the axons. When placing the pipettes transverse with respect to the midline of the nerve, the current may take a more direct path through the axonal tissue.

The choice of laser is also a contributor to the reproducibility of hybrid stimulation. The two lasers used in this study differ in many respects, but are expected to perform equally from the point of view of thermal laser-tissue interaction. However, the Ho:YAG laser yielded greater reproducibility in the rat than did the Capella. To understand how this may have occurred, the two laser sources were examined. The Capella used for this study is a diode laser, which is chopped to produce square pulses having tunable pulse duration at a center wavelength of 1.875 μm. The Ho:YAG laser is a pulsed solid-state laser at 2.12 μm, which produces a 250 μs pulse (full width at half maximum), exhibiting an initial rising phase followed by a decay, with spikes in output energy throughout the pulse duration. The mechanism by which pulsed infrared light produces neural activation is known to be thermally mediated, and directly associated with the absorption of infrared light by water in tissue [52]. Which attribute of the laser contributes most significantly to the thermal gradient is the most relevant issue. A comparison of the absorption coefficient as a function of wavelength for pure water reveals that 1.875 μm and 2.12 μm have similar absorption coefficients (μₐ=26.9 cm⁻¹ and μₐ=24.01 cm⁻¹, respectively) [65]. Although tissue is predominantly water, these values may differ slightly in our preparation and are known to be temperature dependent. However, it is unlikely that the differing wavelengths of the lasers is the source of the Ho:YAG laser's superior reproducibility in myelinated peripheral nerves. A second obvious difference is the pulse durations of the two lasers. However, there is conflicting evidence as to whether pulse duration plays a role in optical stimulation thresholds [52, 67]. A third possibility is that the broad spectral width of the Capella (15-20 nm, FWHM) causes much of the laser's output to occur at wavelengths that are not optimal for optical stimulation of peripheral, myelinated nerves. In applications with more direct access to the target neural tissue, the effects of spectral width are minimized due to all of the light being absorbed at the site of neuronal activation. However, in peripheral nerves, where the optical energy must penetrate through connective tissue and myelin surrounding the axons, longer wavelengths emitted by the Capella may be absorbed before they ever reach the axons. Thus, stimulation thresholds would be higher and quickly approach damage thresholds. The differing temporal pulse structure has not been investigated, but may also contribute to the relative effectiveness of the lasers. Whereas the Capella is a chopped diode laser exhibiting a square pulse, the Ho:YAG laser has a temporal structure in which the optical energy varies and includes numerous energy spikes throughout the pulse duration [74]. This could result in higher peak power and peak irradiance for the Ho:YAG laser.

There are two broad categories of factors that affect the reproducibility of hybrid stimulation related to the interaction of the optical and electrical stimuli. In the first category are spatial factors, where the relative location of the two stimuli determines the efficacy of stimulation. The initial working hypothesis was that for a given sub-threshold radiant exposure, hybrid stimulation would be possible for all locations between the cathode and anode of a bipolar stimulus. The results of this study have shown that hypothesis to be false. In FIG. 12, it is clear that there is a finite ROE for the combination of a constant sub-threshold radiant exposure delivered simultaneously with an electrical stimulus that is 90% of electrical stimulation threshold. While FIG. 12 is drawn from data in the Aplysia buccal nerve, FIG. 13 shows that the same results were seen in the rat sciatic nerve as well. Therefore, successful and reproducible hybrid stimulation calls for accurate placement of the optical fiber relative to the site of electrical stimulation.

This raises the question of where the ROE is located. This answer is clearer in Aplysia, where the ROE was consistently located adjacent to the cathode. Within a single nerve, the location of the ROE was effectively ‘steered’ by reversing the polarity of the electrical stimulus. In the rat sciatic nerve, half of the nerves showed a statistically significant shift in ROE location upon polarity reversal, though the effect was not as dramatic as in Aplysia. In the other trials, the ROE location either did not shift, or hybrid stimulation was ineffective when the polarity was reversed. However, in cases of successful hybrid stimulation, different evoked potentials were recruited for each stimulus polarity. This suggests that hybrid stimulation offers two forms of selectivity, as both the position of the optical stimulus and the polarity of the electrical stimulus dictate the units recruited. The results also imply that the ROE location in the rat sciatic nerve is influenced more by whether or not optical stimulation is possible rather than by the direction of current flow. Anecdotal evidence reveals that there are ‘sweet spots’ on the sciatic nerve where optical stimulation is most effective; in particular, these spots are found just proximal to the branch point of the fascicles, but also at some additional locations along the nerve trunk. This could potentially be due to thinning of the epineurium, proximity of fascicles to the irradiated surface or to increased concentration of nodes of Ranvier in these locations.

The existence of a finite ROE with the potential for shifting location in response to polarity reversal must be taken into account for reproducible hybrid stimulation. Much of the previously observed variability is also likely to be due to the relationship between ROE size and applied radiant exposure. The results indicate an approximately linear increase in ROE size over the range of radiant exposures tested (FIG. 13F). Thus, the center of the ROE will have the lowest threshold radiant exposures when combined with a given sub-threshold electrical stimulus. If this is not accounted for (as was the case in [49]), the variability in the measured thresholds is certainly expected. Furthermore, with the highest probability of firing at the center (FIG. 14), it is likely that an optical stimulus located along the periphery of the ROE induces a reduced firing rate.

A second category of factors contributing to the reproducibility of hybrid stimulation is temporal factors. These factors include how the electrical stimulation threshold and the hybrid stimulation RE₅₀ change with time and relative to one another. It was initially expected that the excitability of a nerve to the combination of electrical and hybrid optical stimuli would follow a similar temporal pattern. However, FIGS. 16 and 17 illustrate a negative correlation between the electrical and hybrid optical stimuli in both Aplysia and rat. If the sub-threshold electrical stimulus is set and the underlying electrical stimulation threshold subsequently decreases (so that an electrical stimulus approaches the stimulation threshold), one would expect the threshold for the optical component of hybrid stimulation to be reduced as well. However, the results did not show this to be true. Thus, one may conclude that the underlying mechanisms of optical and electrical stimulation are dissimilar. If the mechanisms were similar, one would expect a positive correlation between thresholds for electrical stimulation and the optical component of hybrid stimulation. Instead, the data show that as the nerve becomes more excitable to electrical stimulation, its excitability in response to optical stimulation decreases. In the rat, an unexpected decay of electrical threshold currents over time was observed (FIG. 17). This decay may be a sign of increased excitability in response to surgery or trauma.

The underlying electrical stimulation threshold must be taken into account to reduce variability and enhance the reproducibility of hybrid stimulation. Whenever short-term fluctuations (minutes) in threshold radiant exposures are present, controlling for these fluctuations yields overall long-term (1 h) threshold radiant exposures that are consistent (FIGS. 16A and 16B). If electrical stimulation threshold is not controlled over time (as the case in [14]), the variability of measured thresholds for the optical component of hybrid stimulation will increase. This is evident in FIG. 16B. When the sub-threshold electrical stimulus was only set to the chosen magnitude every 20 min, the threshold for the optical component of hybrid stimulation increased and its 95% confidence interval (indicative of the variability) showed greater than a twofold increase. It should be noted that while the inter-rat variability represented in FIG. 17B is much greater than in Aplysia (FIG. 16B), the overall variability and reduction in INS threshold are much lower than what was previously reported. Taking the minimum bound of the 95% RE₅₀ confidence interval for animal 1 and the maximum bound for animal 2 yields an RE₅₀ for hybrid stimulation ranging from 12% to 29% of the radiant exposures required for optical stimulation alone, as opposed to the roughly 30-80% in the previous study.

In the course of investigating temporal factors affecting hybrid stimulation, it was discovered that elevated radiant exposures (although still below threshold radiant exposures for optical stimulation alone) resulted in a decline in the probability of firing (FIG. 18). Sub-threshold radiant exposures for optical stimulation alone were also shown to inhibit electrically evoked potentials (FIG. 19). These results indicate that the potential exists for full hybrid electro-optical control of neural tissue, making it possible to selectively excite or inhibit axons. Preliminary results indicate a spatially confined region of inhibition surrounded by excitation (either hybrid or electrically evoked), suggesting that this is not an artifact, but is a spatially discrete phenomenon, although it may be due to a different mechanism than the excitatory effect. Without an elucidated mechanism of INS, it is difficult to conclude how pulsed infrared light inhibits electrically evoked potentials. Recently, it was shown that intracellular calcium increases in response to optical stimulation of cardiomyocytes [61]. It is conceivable that for hybrid stimulation, supra-threshold radiant exposures may cause an increase in intracellular calcium that activates calcium-dependent potassium channels, thus hyperpolarizing the cell. Further studies will be required to test this hypothesis.

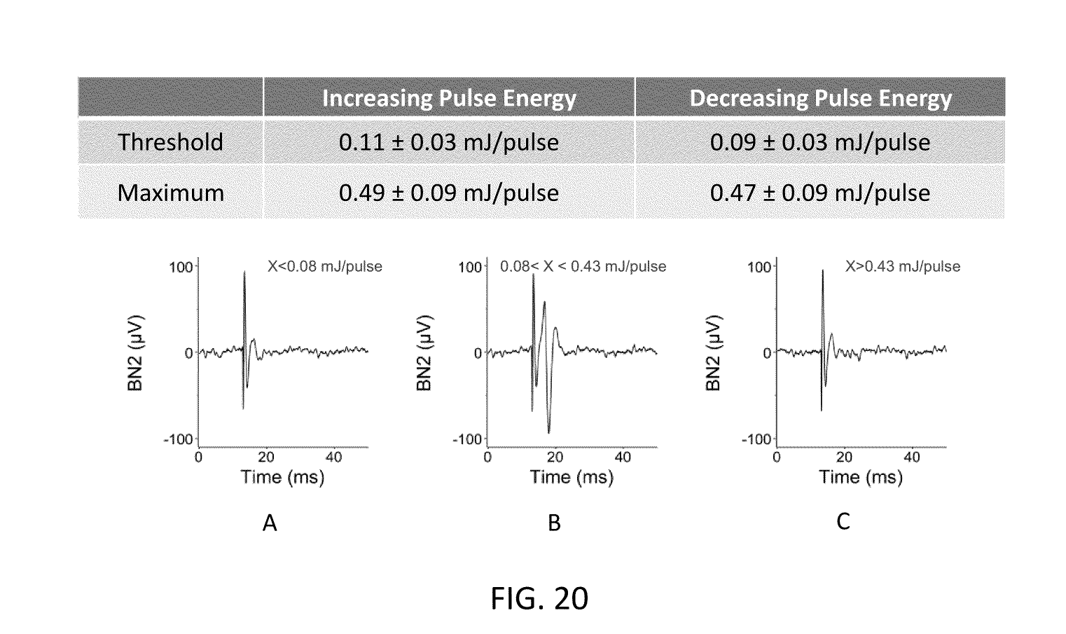

We previously showed the proof-of-concept potential for combined optical and electrical stimulation of neural tissue [49]. This study extends that work by outlining some potential sources of variability that may be controlled to provide reproducible hybrid stimulation. The results presented here also demonstrate the potential of combining optical and electrical stimulation techniques by providing further evidence for selectivity as well as the ability to inhibit neuronal firing. Finally, the study demonstrates the translational value of parallel studies in invertebrates and vertebrates. The key aspects of the methodology to capitalize on the potential of hybrid electro-optical stimulation are summarized as follows.
The optical stimulus, electrical stimulus and target tissue should be mechanically stabilized and controlled relative to one another.
The laser and target neural anatomy must be taken into account to determine the maximum possible expected reproducibility.
For constant electrical priming current, the optical stimulus must be located within the ROE.
For constant electrical priming current, the size of the ROE depends on the strength of the optical stimulus.
Variability in the electrical stimulation threshold induces variability in the RE₅₀ for hybrid stimulation. This variability can be reduced by frequent adjustments to maintain a constant sub-threshold electrical stimulus relative to the electrical stimulation threshold.
There is a range of radiant exposures for which hybrid stimulation has >50% probability of firing. Radiant exposures below or above this range have <50% probability of firing (FIG. 20).

Having taken these points into account, the efficacy is improved by threefold in both the Aplysia californica buccal nerve and the rat sciatic nerve. There are other potential sources of variability that could be controlled to bring the current efficacy up to 100%. In Aplysia, the three nerves that did not show hybrid stimulation were from animals with questionable health, but were included in the success rate calculations for completeness. In myelinated peripheral nerves, the efficacy of optical stimulation is crucial to the success of hybrid stimulation. Elucidating the mechanism of INS will provide a priori knowledge of where on the nerves to stimulate (e.g., near the nodes of Ranvier). Improving the efficacy of optical stimulation in turn improves the efficacy and reduce variability of hybrid stimulation. Knowing the mechanism of INS also provides a clearer understanding of the interaction between electrical and optical stimuli that drives hybrid stimulation. In this study, it was demonstrated that mechanical stabilization of the nerve, electrodes and optical fiber is of utmost importance. Even with the efforts taken to stabilize the system, there is potentially still movement-inducing variability. To address this issue, we envision a hybrid stimulation cuff that moves with the nerve and is thus able to hold the stimuli in place relative to the nerve. However, the results thus far have provided the ability to begin assessing the clinical utility of hybrid neural stimulation. It is believed that the concepts and techniques presented in this study will facilitate the application of spatially selective neural interfaces where thermal tissue damage and/or laser design constraints are currently of concern.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### REFERENCE LIST

[1]. K. L. Kilgore, N. Bhadra, Nerve conduction block utilising high-frequency alternating current. Med Biol Eng Comput 42, 394 (May, 2004).
[2]. K. Deisseroth, Optogenetics. Nat Methods 8, 26 (January, 2011).
[3]. R. T. LaLumiere, A new technique for controlling the brain: optogenetics and its potential for use in research and the clinic. Brain Stimul 4, 1 (January, 2011).
[4]. J. Wells, C. Kao, E. D. Jansen, P. Konrad, A. Mahadevan-Jansen, Application of infrared light for in vivo neural stimulation. Journal of Biomedical Optics 10, (November-December, 2005).
[5]. J. Wells et al., Biophysical mechanisms of transient optical stimulation of peripheral nerve. Biophysical Journal 93, 2567 (October, 2007).
[6]. M. G. Shapiro, K. Homma, S. Villarreal, C. P. Richter, F. Bezanilla, Infrared light excites cells by changing their electrical capacitance. Nat Commun 3, 736 (2012).
[7]. N. M. Fried, G. A. Lagoda, N. J. Scott, L. M. Su, A. L. Burnett, Noncontact stimulation of the cavernous nerves in the rat prostate using a tunable-wavelength thulium fiber laser. J Endourol 22, 409 (March, 2008).
[8]. I. U. Teudt, A. E. Nevel, A. D. Izzo, J. T. Walsh, Jr., C. P. Richter, Optical stimulation of the facial nerve: a new monitoring technique? Laryngoscope 117, 1641 (September, 2007).
[9]. J. M. Cayce, R. M. Friedman, E. D. Jansen, A. Mahavaden-Jansen, A. W. Roe, Pulsed infrared light alters neural activity in rat somatosensory cortex in vivo. Neuroimage 57, 155 (Jul. 1, 2011).
[10]. A. D. Izzo, C. P. Richter, E. D. Jansen, J. T. Walsh, Laser stimulation of the auditory nerve. Lasers in Surgery and Medicine 38, 745 (September, 2006).
[11]. G. M. Dittami, S. M. Rajguru, R. A. Lasher, R. W. Hitchcock, R. D. Rabbitt, Intracellular calcium transients evoked by pulsed infrared radiation in neonatal cardiomyocytes. J Physiol 589, 1295 (Mar. 15, 2011).
[12]. M. W. Jenkins et al., Optical pacing of the embryonic heart. Nat Photonics 4, 623 (Aug. 15, 2010).
[13]. A. R. Duke et al., Combined optical and electrical stimulation of neural tissue in vivo. Journal of Biomedical Optics 14, 060501 (2009).
[14]. A. R. Duke, H. Lu, M. W. Jenkins, H. J. Chiel, E. D. Jansen, Spatial and temporal variability in response to hybrid electro-optical stimulation. J Neural Eng 9, 036003 (Apr. 16, 2012).
[15]. A. L. Hodgkin, B. Katz, The effect of temperature on the electrical activity of the giant axon of the squid. J Physiol 109, 240 (August, 1949).
[16]. F. Rattay, M. Aberham, Modeling axon membranes for functional electrical stimulation. IEEE Trans Biomed Eng 40, 1201 (December, 1993).
[17]. Z. Mou, I. F. Triantis, V. M. Woods, C. Toumazou, K. Nikolic, A simulation study of the combined thermoelectric extracellular stimulation of the sciatic nerve of the Xenopus laevis: the localized transient heat block. IEEE Trans Biomed Eng 59, 1758 (June, 2012).
[18]. A. F. Huxley, Ion movements during nerve activity. Ann N Y Acad Sci 81, 221 (Aug. 28, 1959).
[19]. E. N. Warman, H. J. Chiel, A new technique for chronic single-unit extracellular recording in freely behaving animals using pipette electrodes. J Neurosci Methods 57, 161 (April, 1995).
[20]. R. Nargeot, D. A. Baxter, J. H. Byrne, Contingent-dependent enhancement of rhythmic motor patterns: an in vitro analog of operant conditioning. J Neurosci 17, 8093 (Nov. 1, 1997).
[21]. C. P. Richter et al., Spread of cochlear excitation during stimulation with pulsed infrared radiation: inferior colliculus measurements. J Neural Eng 8, 056006 (October, 2011).
[22]. J. Wells, P. Konrad, C. Kao, E. D. Jansen, A. Mahadevan-Jansen, Pulsed laser versus electrical energy for peripheral nerve stimulation. J Neurosci Methods 163, 326 (Jul. 30, 2007).
[23]. M. E. Llewellyn, K. R. Thompson, K. Deisseroth, S. L. Delp, Orderly recruitment of motor units under optical control in vivo. Nat Med 16, 1161 (October, 2010).
[24]. D. M. Ackermann, Jr. et al., Electrical conduction block in large nerves: high-frequency current delivery in the nonhuman primate. Muscle Nerve 43, 897 (June, 2011).
[25]. D. M. Ackermann, E. L. Foldes, N. Bhadra, K. L. Kilgore, Nerve conduction block using combined thermoelectric cooling and high frequency electrical stimulation. J Neurosci Methods 193, 72 (Oct. 30, 2010).
[26]. M. Dummer et al. (2011), vol. 7883, pp. 85.
[27]. M. Hibbs-Brenner, K. Johnson, M. Bendett. (2009), vol. 7180, pp. 71800-71810.
[28]. S. Thomsen, Pathologic analysis of photothermal and photomechanical effects of laser-tissue interactions. Photochem Photobiol 53, 825 (June, 1991).
[29]. R. Anderson, E. Ross, Laser-tissue interactions. Cutaneous Laser Surgery. Mosby: Philadelphia, Pa. 9, (1994).
[30]. A. L. Hodgkin, Evidence for electrical transmission in nerve: Part I. J Physiol 90, 183 (Jul. 15, 1937).
[31]. G. M. Hale, M. R. Querry, Optical Constants of Water in the 200-nm to 200-microm Wavelength Region. Appl Opt 12, 555 (Mar. 1, 1973).
[32]. J. M. Khosrofian, B. A. Garetz, Measurement of a Gaussian Laser-Beam Diameter through the Direct Inversion of Knife-Edge Data. Applied Optics 22, 3406 (1983).
[33]. S. C. Rosen, M. W. Miller, C. G. Evans, E. C. Cropper, I. Kupfermann, Diverse synaptic connections between peptidergic radula mechanoafferent neurons and neurons in the feeding system of Aplysia. J Neurophysiol 83, 1605 (March, 2000).
[34]. A. R. Duke, H. Lu, M. W. Jenkins, E. D. Jansen, H. J. Chiel, paper presented at the BIOS: Biomedical Optics, Photons and Neurons IV, SPIE Photonics West, San Francisco, Calif., 2012.
[35]. T. J. Pfefer et al., Bioheat transfer analysis of cryogen spray cooling during laser treatment of port wine stains. Lasers Surg Med 26, 145 (2000).
[36]. Tyler D J and Durand D M 2002 Functionally selective peripheral nerve stimulation with a flat interface nerve electrode IEEE Trans. Neural Syst. Rehabil. Eng. 10 294-303.
[37]. Tyler D J and Durand D M 2003 Chronic response of the rat sciatic nerve to the flat interface nerve electrode Ann. Biomed. Eng. 31 633-42.
[38]. Branner A and Normann R A 2000 A multielectrode array for intrafascicular recording and stimulation in sciatic nerve of cats Brain Res. Bull. 51 293-306.
[39]. Malmstrom J A, McNaughton T G and Horch K W 1998 Recording properties and biocompatibility of chronically implanted polymer-based intrafascicular electrodes Ann. Biomed. Eng. 26 1055-64.
[40]. Allegre G, Avrillier S and Albe-Fessard D 1994 Stimulation in the rat of a nerve fiber bundle by a short UV pulse from an excimer laser Neurosci. Lett. 180 261-4.
[41]. Arvanitaki A and Chalazonitis N 1961 Excitatory and inhibitory processes initiated by light and infra-red radiations in single identifiable nerve cells Nervous Inhibition ed E Florey (New York: Pergamon).
[42]. Booth J, von M A and Stampfli R 1950 The photochemical action of ultra-violet light on isolated single nerve fibres Helv. Physiol. Pharmacol. Acta 8 110-27.
[43]. Fork R L 1971 Laser stimulation of nerve cells in Aplysia Science 171 907-8.
[44]. Adams S R and Tsien R Y 1993 Controlling cell chemistry with caged compounds Annu. Rev. Physiol. 55 755-84.
[45]. Boyden E S, Zhang F, Bamberg E, Nagel G and Deisseroth K 2005 Millisecond-timescale, genetically targeted optical control of neural activity Nature Neurosci. 8 1263-8.
[46]. Wells J, Kao C, Mariappan K, Albea J, Jansen E D, Konrad P and Mahadevan-Jansen A 2005 Optical stimulation of neural tissue in vivo Opt. Lett. 30 504-6.
[47]. Wells J, Konrad P, Kao C, Jansen E D and Mahadevan-Jansen A 2007 Pulsed laser versus electrical energy for peripheral nerve stimulation J. Neurosci. Methods 163 326-37.
[48]. Wells J D, Thomsen S, Whitaker P, Jansen E D, Kao C C, Konrad P E and Mahadevan-Jansen A 2007 Optically mediated nerve stimulation: identification of injury thresholds Lasers Surg. Med. 39 513-26.
[49]. Duke A R, Cayce J M, Malphrus J D, Konrad P, Mahadevan-Jansen A and Jansen E D 2009 Combined optical and electrical stimulation of neural tissue in vivo J. Biomed. Opt. 14 060501.
[50]. Elliott C J H and Susswein A J 2002 Comparative neuroethology of feeding control in molluscs J. Exp. Biol. 205 877-96.
[51]. Kandel E R 1979 Behavioral Biology of Aplysia: A Contribution to the Comparative Study of Opisthobranch Molluscs (San Francisco: Freeman).
[52]. Wells J, Kao C, Konrad P, Milner T, Kim J, Mahadevan-Jansen A and Jansen E D 2007 Biophysical mechanisms of transient optical stimulation of peripheral nerve Biophys. J. 93 2567-80.
[53]. Holsheimer J 2003 Principles of neurostimulation Pain Res. Clin. Manage. 15 17-36.
[54]. Rattay F 1986 Analysis of models for external stimulation of axons IEEE Trans. Biomed. Eng. 33 974-7.
[55]. Ranck J B Jr 1975 Which elements are excited in electrical stimulation of mammalian central nervous system: a review Brain Res. 98 417-40.
[56]. Izzo A D, Walsh J T, Ralph H, Webb J, Bendett M, Wells J and Richter C P 2008 Laser stimulation of auditory neurons: effect of shorter pulse duration and penetration depth Biophys. J. 94 3159-66.
[57]. Blair E A and Erlanger J 1935 On the process of excitation by brief shocks in axons Am. J. Physiol. 114 309
[58]. Teudt I U, Nevel A E, Izzo A D, Walsh J T Jr and Richter C P 2007 Optical stimulation of the facial nerve: a new monitoring technique? Laryngoscope 117 1641-7.
[59]. Wells J, Kao C, Jansen E D, Konrad P and Mahadevan-Jansen A 2005 Application of infrared light for in vivo neural stimulation J. Biomed. Opt. 10 064003.
[60]. Cayce J M, Friedman R M, Jansen E D, Mahavaden-Jansen A and Roe A W 2011 Pulsed infrared light alters neural activity in rat somatosensory cortex in vivo Neuroimage 57 155-66.
[61]. Dittami G M, Rajguru S M, Lasher R A, Hitchcock R W and Rabbitt R D 2011 Intracellular calcium transients evoked by pulsed infrared radiation in neonatal cardiomyocytes J. Physiol. 589 1295-306.
[62]. Harris D M, Bierer S M, Wells J D and Phillips J 0 2009 Optical nerve stimulation for a vestibular prosthesis Proc. SPIE 7180 71800R.
[63]. Izzo A D, Suh E, Pathria J, Walsh J T, Whitlon D S and Richter C P 2007 Selectivity of neural stimulation in the auditory system: a comparison of optic and electric stimuli J. Biomed. Opt. 12 021008.
[64]. Jenkins M W, Duke A R, Gu S, Chiel H J, Fujioka H, Watanabe M, Jansen E D and Rollins A M 2010 Optical pacing of the embryonic heart Nature Photon. 4 623-6.
[65]. Hale G M and Querry M R 1973 Optical constants of water in the 200 nm to 200-microm wavelength region Appl. Opt. 12 555-63.
[66]. Khosrofian J M and Garetz B A 1983 Measurement of a Gaussian laser-beam diameter through the direct inversion of knife-edge data Appl. Opt. 22 3406-10.
[67]. Izzo A D, Walsh J T Jr, Jansen E D, Bendett M, Webb J, Ralph H and Richter C P 2007 Optical parameter variability in laser nerve stimulation: a study of pulse duration, repetition rate, and wavelength IEEE Trans. Biomed. Eng. 54 1108-14.
[68]. Gibson J M and Welker W 11983 Quantitative studies of stimulus coding in first-order vibrissa afferents of rats: II. Adaptation and coding of stimulus parameters Somatosens. Res. 1 95-117.
[69]. Hermann M, Hellebart C and Freissmuth M 2004 Neuromonitoring in thyroid surgery: prospective evaluation of intraoperative electrophysiological responses for the prediction of recurrent laryngeal nerve injury Ann. Surg. 240 9-17.
[70]. Julkunen P, Ruohonen J, Saaskilahti S, Saisanen L and Karhu J 2011 Threshold curves for transcranial magnetic stimulation to improve reliability of motor pathway status assessment Clin. Neurophysiol. 122 975-83.
[71]. Kirby A E and Middlebrooks J C 2010 Auditory temporal acuity probed with cochlear implant stimulation and cortical recording J. Neurophysiol. 103 531-42.
[72]. Butson C R, Miller 10, Normann R A and Clark G A 2011 Selective neural activation in a histologically derived model of peripheral nerve J. Neural Eng. 8 036009.
[73]. Johnston W L, Dyer J R, Castellucci V F and Dunn R J 1996 Clustered voltage-gated Na+ channels in Aplysia axons J. Neurosci. 16 1730-9.
[74]. Asshauer T, Delacretaz G, Jansen E, Welch A and Frenz M 1997 Pulsed holmium laser ablation of tissue phantoms: correlation between bubble formation and acoustic transients Appl. Phys. B 65 647-57.

What is claimed is:

1. A method of transient and selective suppression of neural activities of a target of interest, comprising:
selectively applying at least one light to the target of interest at selected locations with predetermined radiant exposures to create a localized and selective inhibitory response therein.

2. The method of claim 1, wherein the target of interest contains one or more nerves.

3. The method of claim 1, wherein the neural activities comprise generation and propagation of action potentials.

4. The method of claim 3, wherein the action potentials are evoked electrically by an electrical stimulus applied to the target of interest.

5. The method of claim 4, wherein the at least one light comprises pulses of a single light generated from a laser source.

6. The method of claim 5, wherein the pulses of the single light are synchronized with the electrical stimulus, such that the pulses of the single light and the electrical stimulus end at the same time.

7. The method of claim 5, wherein the pulses of the single light are applied prior to the start time of the electrical stimulus at a first predetermined time.

8. The method of claim 5, wherein the pulses of the single light are applied after the start time of the electrical stimulus at a second predetermined time.

9. The method of claim 4, wherein the at least one light comprises two or more lights, wherein each of the two or more lights comprises pulses of light generated from a respective laser source.

10. The method of claim 9, wherein the pulses of the two or more lights are synchronized with the electrical stimulus, such that the pulses of the two or more lights and the electrical stimulus end at the same time.

11. The method of claim 9, wherein the pulses of the two or more lights are applied prior to the start time of the electrical stimulus at a first predetermined time.

12. The method of claim 9, wherein the pulses of the two or more lights are applied after the start time of the electrical stimulus at a second predetermined time.

13. The method of claim 9, wherein the step of selectively applying the at least one light to the target of interest comprises:
simultaneously applying the two or more lights to the target of interest at the selected locations,

14. The method of claim 9, wherein the step of selectively applying the at least one light to the target of interest comprises:
alternately or sequentially applying the two or more lights to the target of interest at the selected locations.

15. The method of claim 1, wherein each of the at least one light comprises an infrared light.

16. The method of claim 1, wherein the localized and selective inhibitory response comprises a local temperature change

17. An apparatus for selectively controlling of neural activities of a target of interest, comprising:
a light source for generating at least one light; and
a probe coupled to the at least one light source for selectively delivering the at least one light to the target of interest at selected locations to create a localized and selective inhibitory response therein.

18. The apparatus of claim 17, wherein the target of interest contains one or more nerves.

19. The apparatus of claim 17, wherein the neural activities comprise generation and propagation of action potentials.

20. The apparatus of claim 19, wherein the action potentials are evoked electrically by an electrical stimulus applied to the target of interest.

21. The apparatus of claim 20, wherein the light source comprises a laser source, and the at least one light comprises pulses of a single light generated from the laser source.

22. The apparatus of claim 21, wherein the pulses of the single light are synchronized with the electrical stimulus, such that the pulses of the single light and the electrical stimulus end at the same time.

23. The apparatus of claim 21, wherein the pulses of the single light are applied prior to the start time of the electrical stimulus at a first predetermined time.

24. The apparatus of claim 21, wherein the pulses of the single light are applied after the start time of the electrical stimulus at a second predetermined time.

25. The apparatus of claim 20, wherein the light source comprises two or more light laser sources, and wherein the at least one light comprises two or more lights, each light comprising pulses of light generated from a respective laser source of the two or more light laser sources.

26. The apparatus of claim 25, wherein the pulses of the two or more lights are synchronized with the electrical stimulus, such that the pulses of the two or more lights and the electrical stimulus end at the same time.

27. The apparatus of claim 25, wherein the pulses of the two or more lights are applied prior to the start time of the electrical stimulus at a first predetermined time.

28. The apparatus of claim 25, wherein the pulses of the two or more lights are applied after the start time of the electrical stimulus at a second predetermined time.

29. The apparatus of claim 25, wherein the probe is configured to simultaneously deliver the two or more lights to the target of interest at the selected locations,

30. The apparatus of claim 25, wherein the probe is configured to alternately or sequentially deliver the two or more lights to the target of interest at the selected locations.

31. The apparatus of claim 17, wherein each of the at least one light comprises an infrared light.

32. The apparatus of claim 17, wherein the probe comprises at least one optical fiber having one end coupled to the at least light source and a working end positioned proximate to the target of interest for selectively delivering the at least one light to the target of interest at the selected locations.

33. A method for identifying spatial factors that are controllable for enhancing reproducibility of a hybrid electro-optical stimulation to a target of interest, comprising:
simultaneously applying electrical pulses at a sub-threshold and optical pulses of a set magnitudes to the target of interest, wherein the optical pulses of a set magnitudes are delivered by an optical fiber;
translating the optical fiber back and forth across the target of interest, and measuring a position of the optical fiber when translating;
reconstructing the exact position of the optical fiber at the time of the hybrid stimulation; and
correlating the working end of the optical fiber with the presence or absence of the hybrid stimulation as indicated by an evoked potential on a nerve recording, so as to obtain the spatial factors.

34. The method of claim 33, wherein the sub-threshold is about 90% less than the threshold of the electrical stimulation.

35. The method of claim 33, further comprising:
determining existence of a finite region of excitability (ROE) with size altered by the strength of the optical stimulus and recruitment dictated by the polarity of the electrical stimulus.

36. The method of claim 33, wherein the electrical pulses and the optical pulses are synchronized such that they end concurrently.

37. A method for identifying temporal factors that are controllable for enhancing reproducibility of a hybrid electro-optical stimulation to a target of interest, comprising:
simultaneously applying electrical pulses and optical pulses to the target of interest;
regularly measuring threshold currents of the electrical stimulus to monitor underlying changes in the electrical stimulation with time, and measuring radiant exposures eliciting the hybrid stimulation along with the threshold currents of the electrical stimulus;
reducing the stimulus current to a sub-threshold;
applying different radiant exposures along with the sub-threshold current pulses to the target of interest, and recording each hybrid stimulus pulse as either a 1 or 0 as determined by the presence (1) or absence (0) of action potentials;
repeating the process for the predetermined duration; and
processing the recorded data to obtain the temporal factors.

38. The method of claim 37, wherein the electrical pulses and the optical pulses are synchronized such that they end concurrently.

39. The method of claim 37, wherein the sub-threshold is about 90% less than the threshold of the electrical stimulation.
